(19) United States
(12) Patent Application Publication
(10) Pub. No.: US 2025/0120372 A1
(43) Pub. Date: Apr. 17, 2025

(54) HUMANIZED MOUSE MODEL OF FIBROSIS

(71) Applicants: The Jackson Laboratory, Bar Harbor, ME (US); University of Massachusetts, Boston, MA (US); Massachusetts Institute of Technology, Cambridge, MA (US)

(72) Inventors: Leonard D. Shultz, Bar Harbor, ME (US); Michael A. Brehm, Boston, MA (US); Dale L. Greiner, Boston, MA (US); Daniel Anderson, Cambridge, MA (US); Joshua C. Doloff, Towson, MD (US)

(73) Assignees: The Jackson Laboratory, Bar Harbor, ME (US); University of Massachusetts, Boston, MA (US); Massachusetts Institute of Technology, Cambridge, MA (US)

(21) Appl. No.: 18/720,575

(22) PCT Filed: Dec. 16, 2022
(86) PCT No.: PCT/US2022/053136

§ 371 Date: Jun. 14, 2024

Related U.S. Application Data
(60) Provisional application No. 63/290,965, filed on Dec. 17, 2021.

(51) Int. Cl. A01K 67/0271; A01K 67/0278

(52) U.S. Cl. CPC A01K 67/0271; A01K 67/0278; A01K 2207/12; A01K 2207/15; A01K 2217/052; A01K 2227/105; A01K 2267/03

(57) ABSTRACT

The present disclosure provides, in some aspects, a humanized immunodeficient mouse model of human fibrosis. A humanized immunodeficient mouse model provided herein may be used, for example, for modeling human fibrosis, predicting human fibrosis, and testing putative fibrosis treatments.

### RELATED APPLICATIONS

This application claims the benefit under 35 U.S.C. § 119(e) of U.S. provisional application No. 63/290,965, filed Dec. 17, 2021, which is incorporated by reference herein in its entirety.

### GOVERNMENT LICENSE RIGHTS

This invention was made with government support under grant numbers UC4 DK104218, R24OD0426640, and R01AI132963 awarded by National Institutes of Health. The government has certain rights in the invention.

### BACKGROUND

Implanted biomedical devices are an integral part of modern medicine, essential for many therapies, and are used in millions of surgeries every year. Nonetheless, they elicit immunogenic responses, which lead to fibrotic encapsulation as part of foreign body rejection response (FBR). Interestingly, the physical architecture, specifically a larger size and spherical shape of implants, abrogates the immune response to multiple classes of materials implanted in rodents and non-human primates. Certain biomaterial surface chemistries and drugs have also been identified to significantly reduce fibrosis in both models as well.

Biocompatibility studies are often carried out in rodents, as there is limited accessibility to non-human primate models, and multiple barriers to human study. Ethical and technical concerns can also make in vivo testing in large animals and humans prohibitive. As a result of immunologic differences between humans and mice, rodent models may not be predictive of the translational potential of biomaterial platforms in human patients. Furthermore, historical pre-clinical screening inconsistencies have arisen in the field of fibrosis due to differences across different strains of mice, such as general lack of fibrosis in BALB/c mice vs. the more substantial fibrotic response in C57BL/6 mice.

### SUMMARY

Provided herein, in some aspects, are humanized mouse models that recapitulate fibrosis following biomaterial implantation. Cellular and cytokine responses to multiple biomaterials were evaluated across different implant sites. Surprisingly, human innate immune macrophages were shown to be essential to biomaterial rejection in this model, and these macrophages were even capable of crosstalk with mouse fibroblasts for collagen matrix deposition. Cytokine and cytokine-receptor array analysis, in some embodiments, confirmed core signaling in the fibrotic cascade. Unexpectedly, foreign-body giant cell formation, often unobserved in mice, was also prominent, in some instances. Lastly, high-resolution microscopy coupled with multiplexed antibody-capture digital profiling analysis supplied spatial resolution of rejection responses, in some embodiments. This model supports investigation of dynamic human immune cell interactions with implantable exogenous biomaterials, for example, and offers a surrogate pre-clinical diagnostic tool for screening biomaterial immunogenicity.

Some aspects of the present disclosure provide a method, comprising: (a) implanting an exogenous biomaterial into an immunodeficient mouse, wherein the mouse comprises human adaptive immune cells and human innate immune cells; and (b) assessing fibrosis following implantation of the biomaterial.

In some embodiments, the immunodeficient mouse lacks mature mouse T cells, B cells, and functional natural killer (NK cells) and is deficient in mouse cytokine signaling.

In some embodiments, the immunodeficient mouse supports engraftment of CD33+ myeloid lineages.

In some embodiments, the immunodeficient mouse is a non-obese diabetic (NOD) mouse.

In some embodiments, the immunodeficient mouse comprises an inactivated Prkdc^{scid} allele and/or an inactivated mouse Il2rg allele.

In some embodiments, the immunodeficient mouse comprises a transgene encoding a human hematopoietic cytokine.

In some embodiments, the transgene encodes human colony stimulating factor 1 (CSF1).

In some embodiments, the transgene encodes a cytokine selected from human stem cell factor (SCF), human granulocyte/macrophage-colony stimulating factor 2 (GM-CSF2), and human interleukin-3 (IL-3).

In some embodiments, the immunodeficient mouse comprises a transgene encoding human SCF, a transgene encoding human GM-CSF2, and a transgene encoding IL-3.

In some embodiments, the immunodeficient mouse is engrafted with human fetal tissue.

In some embodiments, the human fetal tissue comprises tissue from human fetal bone marrow, liver, and/or thymus.

In some embodiments, the immunodeficient mouse is engrafted with human hematopoietic stem cells.

In some embodiments, the human fetal tissue is autologous with the human hematopoietic stem cells.

In some embodiments, the immunodeficient mice comprise human myeloid cells.

In some embodiments, the immunodeficient mice comprise human macrophages.

In some embodiments, the biomaterial is a synthetic biomaterial.

In some embodiments, the biomaterial comprises hydrogel alginate or polymer polydimethylsiloxane (PDMS).

In some embodiments, the biomaterial is a biomedical device.

In some embodiments, the biomaterial is implanted subcutaneously or intraperitoneally.

In some embodiments, the assessing fibrosis occurs about 1 to 4 weeks following implantation of the biomaterial.

### BRIEF DESCRIPTION OF THE DRAWINGS

FIGS. 1A-1F. Identifying a humanized model capable of biomaterial fibrosis. SLG20 alginate 500 μm diameter spheres (“alginate spheres,” 0.5 ml total implant volume) were implanted into the intraperitoneal (IP) space of pro-fibrotic C57BL/6 mice and various humanized mouse models, retained for 28 days, and analyzed for degree of fibrosis upon alginate sphere retrieval. FIG. 1A is a schematic of humanization to achieve fibrotic foreign body response (FBR) involving a human hematopoietic stem cell (hHSC)-engrafted NSG-SGM3 BLT mouse model. FIG. 1B shows brightfield images obtained from retrieved alginate spheres in control C57BL/6 mice (left), NSG BLT mice, without and with xenogeneic neonatal pig cell clusters (NPCCs) (middle). Successful fibrotic FBR was observed after transgenic expression of 3 human cytokines (IL3, GM-CSF, and SCF in the NSG-SGM3 BLT model, right). FIG. 1C shows additional high magnification (10× and 4×) brightfield images of fibrosed alginate spheres retrieved from engrafted NSG-SGM3 BLT mice 28 days after implantation. FIG. 1D shows phase contrast images of fibrosis in epididymal fat-embedded polymer polydimethylsiloxane (PDMS) 5 mm discs (left) and 500 μm polystyrene spheres (PS, right) retrieved 28 days after intraperitoneal (IP) implantation. FIGS. 1E and 1F show flow cytometry analysis of CD45⁺ (FIG. 1E) and CD33⁺ (FIG. 1F) immune engraftment without human SGM3 cytokine expression (circles) vs. with human SGM3 cytokine expression (squares) in blood, spleen, bone marrow (BM), peritoneal exudative cells (PEC) compartments. Flow statistical analysis: one-way ANOVA with Bonferroni multiple comparison correction ***: p<0.0001; ns=not significantly different. n=5 mice per group. Experiments repeated at least 3 times.

FIGS. 2A-2E. Foreign body rejection response (FBR) human macrophage dependance and mouse fibroblast crosstalk confirmed in NSG-SGM3 BLT humanized model. FIG. 2A shows clodrosome depletion (macro depletion) of human macrophages in non-fibrosed (non-engrafted) and fibrosed (engrafted) controls, which in complete fibrosis loss following alginate 500 μm diameter sphere (Blank capsules) 2-week IP implantations in engrafted NSG-SGM3 BLT mice (“Humanized”). FIG. 2B shows flow cytometry analysis of human cells dissociated from retrieved alginate spheres 2 and 4 weeks post-IP implantation (“2 wk”, “4 wk”, squares and triangles, respectively) showing significantly increasing levels of CD45⁺CD33⁺ myeloid cells, which were also eliminated following clodrosome-maintained macrophage depletion throughout 2-week implantations (“2 wk-Mφ”). FIG. 2C shows flow cytometry analysis of residual mouse macrophages (Cd11b⁺F4/80⁺) and neutrophils (Cd11b⁺/GR1⁺) dissociated from retrieved alginate spheres 2 and 4 weeks post-IP implantation (“2 wk”, “4 wk”, squares and triangles, respectively), and following clodrosome-maintained macrophage depletion throughout 2-week implantations (“2 wk-Mφ”). FIGS. 2D and 2E show NanoString analysis of physical cell and cytokine markers analyzed from deposited cell RNA extracts from alginate spheres collected at 1, 5, and 14 days post-implantation (D1, D5, D14) in engrafted NSG-SGM3 BLT mice, as compared to non-engrafted controls and NSG-SGM3 BLT engrafted mice treated with clodrosomes (My). White, within background of the assay. For all analyses n=5 per treatment. Flow experiments were performed twice and NanoString analysis was performed once. Flow comparisons were performed by one-way ANOVA, ***: p<0.0001, vs clodrosome-depleted controls. NanoString: Log base-2 scale; for stat analyses see methods.

FIGS. 3A-3E. FBR and foreign giant cell formation is observed in the subcutaneous site in engrafted NSG-SGM3 BLT humanized mice. SLG20 alginate (“alginate”) or polystyrene 500 m spheres (0.5 ml), were implanted for 2 weeks or 4 weeks into the subcutaneous space of NSG-SGM3 BLT humanized mice, and the degree of fibrosis was analyzed upon retrieval. FIG. 3A shows hematoxylin and eosin (H&E) and Masson's Trichrome (Masson's) stained histological sections of excised SC tissue 2 weeks and 4 weeks days post-implant for alginate and 14 days for polystyrene (additional photos in FIGS. 7, 10, and 11) (Scale bar=500 μm). FIG. 3B shows confocal microscopy of prominent foreign body giant cell (FBGC) formation (by macrophage marker CD68) co-localized with an inner ring of myofibroblast marker alpha-SM actin (aSMactin). FIG. 3C shows photos of prominent encapsulation of alginate and polystyrene spheres upon retrieval following 4-week SC implantations. FIG. 3D shows confocal microscopy confirming that the majority of earlier-identified (FIGS. 1 and 2) CD33⁺ myeloid cells co-express macrophage marker CD68. FIG. 3E shows NanoString analysis of physical cell and cytokine markers analyzed from deposited cell RNA extracts from polystyrene (PS) spheres and 5 mm PDMS discs collected at 14 and 28 days (d14 and d28) post IP or SC implantations in engrafted NSG-SGM3 BLT mice, as compared to non-engrafted controls. White, within assay background. NanoString analysis was performed once. Log base-2 scale; for stat analyses see methods. n=5 mice/group for all assays. Experiments repeated at least 2-3 times.

FIGS. 4A-4E. Spatial profiling of human immune FBR by digital profiling of immune cells on and around implanted biomaterials in engrafted NSG-SGM3 BLT mice. SLG20 alginate or polystyrene 500 μm diameter spheres (0.5 ml) were implanted into the subcutaneous (SC) space of NSG-SGM3 BLT humanized mice for 14- and 28-days. FIG. 4A Per NanoString digital spatial profiling (DSP) immunofluorescence microscopy using DAPI nuclear (blue), leukocyte marker CD45 (red), and macrophage marker CD68 (green) staining to identify (whole) regions of interest (ROIs) (lined areas). FIG. 4B ROI analysis performed around the same implant site but with successive rounds of ablation where the UV laser was incrementally increased 15 microns at a time, thereby capturing concentric rings of antibody binding and immune response data moving away from the implant surface. FIG. 4C shows heat maps depicting all data for the DSP NanoString human 30-plex antibody panel, with data for both 2-week and 4-week Hydrogel Alginate and Polystyrene implants. FIG. 4D depicts a log-scale line graph depiction of antibody levels (Protein counts) (as reflected in FIG. 4C) as a function of distinct from the implant surface over time. FIG. 4E depicts a log-scale line graph depicting human immune cells levels as a function of distance from the implant surface, where macrophage CD68 was largely localized at the implant surface (see also FIG. 13), while B cell markers CD19 and CD20 decreased more slowly moving away from the implant surface. One-way ANOVA with Bonferroni multiple comparison correction: *: p<0.05 and ***: p<0.0001, vs 0 um distance. ns=not significantly different. n=5 mice/group. NanoString Digital Spatial Profiling experimental analysis run once across n=2 samples per treatment group (SLG20 and Polystyrene sphere implantation) per time point (2 and 4 weeks), with up to 20 areas of interest (green boxes) per sample.

FIGS. 5A-5B. Transgenic delivery of 3 Human cytokines (IL3, GM-CSF, and SCF) enabled fibrosis in the engrafted NSG-SGM3 BLT model. FIG. 5A shows brightfield images of SLG20 alginate 500 μm diameter spheres retrieved 2 weeks after implantation into the intraperitoneal (IP) space of C57BL/6 mice. FIG. 5B shows brightfield images of similar SLG20 alginate spheres retrieved 2 weeks (left) or 4 weeks (right) after IP implantations in the NSG-SGM3 BLT model. Photos representative from n=5 mice per treatment. Experiment run at least twice.

FIGS. 6A-6C. SGM3 cytokine modification of humanized mice increased CD45+ immune cells in the IP space and innate myeloid CD33⁺ immune cells in both the spleen and IP space of engrafted mice. FIG. 6A shows flow cytometry analysis of IP lavaged CD45⁺ peritoneal exudate cells (PECs) in either NSG (circles) or NSG-SGM3 BLT (“SGM3”, squares) mice. FIG. 6B shows flow cytometry analysis of IP lavaged CD45⁺ (top) and CD33⁺ (bottom) spleen cells in either NSG (circles) or NSG-SGM3 BLT (squares) mice. FIG. 6C shows flow cytometry analysis of IP lavaged CD33⁺ peritoneal exudate cells (PECs) in either NSG (circles) or NSG-SGM3 BLT (“SGM3”, squares) mice. Lines, mean. n=5 mice per treatment. Performed at least two times.

FIGS. 7A-7B. SGM3 modification of engrafted humanized mice results in macrophage enrichment across multiple compartments of the intraperitoneal space. FIG. 7A shows flow cytometry of site-specific (intraperitoneal in IP lavage, from IP implanted capsules “Capsules”, or isolated epididymal fat “Epid. Fat”) increases in mature human CD45⁺CD33⁺ myeloid macrophages after 2 or 4 week implantations (“2 wk” and “4 wk” squares and triangles, respectively) versus clodrosome-maintained macrophage depletion after a 2 week implantation (“2 wk-M”, circles) in engrafted NSG-SGM3 BLT mice. FIG. 7B shows flow cytometry of CD45⁺CD14⁺ monocytic infiltration after 2 or 4 week implantations (“2 wk clodrosome”, “2 wk”, “4 wk”; circles, squares, and triangles, respectively). n=5 mice/group. The same material volume of hydrogel spheres was implanted into each mouse in all cases. Experiments were repeated twice.

FIGS. 8A-8B. Histological analysis (H&E and Masson's Trichrome) showing recapitulated fibrotic response in the subcutaneous (SC) implant site. 500 μm diameter alginate (FIG. 8A, “Alginate”) or polystyrene (FIG. 8B) spheres were retrieved from the subcutaneous (SC) space of engrafted NSG-SGM3 BLT mice following either 2- or 4-week implantations (as noted). Images, representative across all mice. Different magnifications (2×, 4×, and 10×) displayed (as noted). Experiment repeated at least twice.

FIGS. 9A-9B. Histological analysis of H&E staining (FIG. 9A) and Masson's Trichrome staining (FIG. 9B) showing developed fibrotic response in the subcutaneous implant site after 4 weeks. 500 μm diameter alginate spheres were retrieved from the subcutaneous (SC) space of humanized NSG-SGM3 BLT mice following 4 week implantations. Images, representative across all mice (n=5/group); Different magnifications (2×, 4×, and 10×) displayed (as noted). Arrows, pointing to examples of remaining alginate spheres. Experiment repeated at least twice.

FIGS. 10A-10B. Example of immunofluorescence staining of subcutaneous (SC) tissue from humanized NSG-SGM3 BLT mice. Immunofluorescent images showing cellular nuclei (DAPI, blue), macrophage marker CD68 (green), or myofibroblast marker (alpha Smooth Muscle actin, aSMactin, red) taken at high 20× (FIG. 10A, bottom) or low 5× (FIG. 10B) magnification. Experiments performed twice.

FIG. 11. Representative brightfield, histology (Masson's Trichrome), and immunofluorescence (IF) panels for subcutaneously (SC) implanted polystyrene spheres in engrafted NSG-SGM3 BLT mice. Samples were first imaged by brightfield, checked by Masson's Trichrome for positive fibrotic encapsulation, and then stained by IF for a general immune marker CD45 (red), a macrophage marker CD68 (green), and a nuclear marker (DAPI, blue). Once positive candidate areas (boxes, right image, up to 20 per sample) were identified digital spatial profiling (DSP) analysis was performed using a 30 antibody-multiplexed probe set was used to determine protein dynamics in the tissue immediately surrounding material implants. Representative images from n=5 mice/group. Experimental analysis run once across n=2 samples per treatment group (SLG20 and Polystyrene sphere implantation) per time point (2 and 4 weeks), with up to 20 areas of interest (boxes, right image) per sample.

FIGS. 12A-12E. Additional selection photos for digital spatial profiling (DSP) analysis. Example brightfield (FIG. 12A) and immunofluorescence (IF) panels (FIG. 12B) for subcutaneously implanted polystyrene spheres in engrafted NSG-SGM3 BLT mice. IF staining: with the immune marker CD45 (red), the macrophage marker CD68 (green), and the nuclear marker (DAPI, blue). FIG. 12C shows an example of one contiguous region of interest (ROI) drawn around one implant sphere for UV ablation and decoupling of nucleotide tags from 30-plex antibody panel. FIG. 12D shows an example of concentric ring ROIs (multiple) where the UV decoupling laser was used incrementally in order to isolate antibody probes in 15 micron (um) wedges starting at the material implant-tissue interface and moving outward and away from the implant surface. FIG. 12E Additional examples of expanded lump sum single ROI vs. concentric ROI-based analyses of immune response in tissue immediately surrounding implanted spheres in fully engrafted NSG-SGM3 BLT mice. Experimental analysis run once across n=2 samples per treatment group (SLG20 alginate sphere and Polystyrene sphere implantation) per time point (2 and 4 weeks), with up to 20 areas of interest (boxes) per sample.

FIG. 13. Digital Spatial Profiling (DSP) to quantify protein levels as a function of distance from implant surfaces. Three line plots (left, middle and right) show quantified protein levels (as denoted) for various immune markers as distance increases away from implant surfaces (in 15 micron concentric rings) for alginate (at 2 weeks post-implant) or polystyrene (2 and 4 weeks post-implant) spheres following subcutaneous retrievals from engrafted humanized NSG-SGM3 BLT mice. Experimental analysis run once across n=2 samples per treatment group (SLG20 and Polystyrene sphere implantation) per time point (2 and 4 weeks), with up to 20 areas of interest (boxes) per sample.

FIG. 14. DSP analysis to quantify protein levels as a function of distance from implant surfaces for tissues from Non-human Primates (NHPs). Samples were stained with H&E for cellular infiltration and Masson's Trichrome for positive fibrotic encapsulation, respectively (FIG. 14A), and then by immunofluorescence (IF) for macrophage marker CD68 (red) and alpha Smooth Muscle actin (SMA, green) (FIG. 14B). Experimental analysis run once across n=2 NHPs.

### DETAILED DESCRIPTION

In an attempt to recapitulate human immune responses observed in patients, humanized mouse models have been developed. These models, of which there are many variants, provide the ability to sample tissues more frequently and to potentially better dissect molecular mechanisms. In such models, mice are modified with human tissues, cells, and/or expressing genes, towards improving the faithfulness with which observed immune responses mirror those in humans. These models have been used as tools to study cancer, tissue inflammation, infectious agents, as well as immune dysregulation. While humanized mice have been under study for the past 30 years, needed improvements in the human immune cell function occurred with the development of immunodeficient IL2rg^{-/-} mice, which have supported heightened levels of immune engraftment¹⁵ and function over earlier humanized NOD-scid models. However, even humanized variants of this model have been criticized for the inability of their immune systems to mimic responses in a human, as numerous differences in immune cell development and maturation as well as lymph node and secondary lymphoid tissue development have been observed between humanized model variants, depending on how they are generated. In the context of biomaterial biocompatibility screening, creation of a humanized screening model has not been reported, as numerous humanized variants exist in the reported literature of tissue rejection, but not biomaterial fibrosis. Provided herein are mouse models generated in part based on the hypothesis that further advances in engraftment, maintenance and behavior of immune cell subsets are required to create humanized mouse models as predictive preclinical tools for fibrosis.

A variety of studies in mice and non-human primate cynomolgus monkeys that mimic FBR observed in humans indicate that innate immune myeloid populations, namely monocyte/macrophages, are required to confer pro-fibrotic ability. Macrophages are a key mediator of material recognition, adhering to the surface of many types of biomaterials, fusing into foreign-body giant cells, prior to myofibroblast induction and fibrous capsule formation. Moreover, certain hematopoietic stem cell (HSC) engrafted humanized models often support adaptive immune B and T cell development, which is ideal for studies in pathogen and transplant response, but not for fibrosis.

The present disclosure provides, in some aspects, a humanized mouse model that engrafts with a robust human innate immune system. This model was used in some embodiments to evaluate the fibrotic response to both naturally and synthetically-derived biomaterials in subcutaneous (SC) and intraperitoneal (IP) implant sites. Of note, naturally derived hydrogel alginate was used, in some examples, given its long history of use as a multipurpose biomaterial that has been evaluated for a range of biomedical applications including biosensors, tissue regeneration, cell encapsulation, and drug delivery, but which has been limited by the fibrotic response as a long-term barrier to their clinical success. Synthetic polymer polydimethylsiloxane (PDMS), relevant for many other biomaterial applications including breast implantation was also used in some examples described herein.

### Methods of Producing Mouse Models of Fibrosis

In some embodiments, the present disclosure provides a model of fibrosis. Fibrosis is development and deposition of connective tissue as a response to tissue injury or damage. Fibrosis may be part of normal healing (e.g., healthy, non-pathologic) or it may be pathologic (e.g., associated with a disease). Pathologic fibrosis may be excess connective tissue deposition above what is required for healing, connective tissue deposition in a tissue area that doesn't have connective tissue and is therefore damaged in the process, or a combination of both. Connective tissue deposition in fibrosis is pathologic because it can replace normal tissue, it can destroy the normal architecture of a tissue, and it can decrease the tissue's ability to function normally (e.g., non-pathologic).

A model of fibrosis herein may be any rodent used to mimic fibrosis. Non-limiting examples of rodents include mice and rats.

Fibrosis may occur in any tissue or organ in a model. Non-limiting examples of tissues or organs that may be affected by fibrosis in a model include: peritoneal cavity, lung (e.g., alveoli, bronchi, bronchioles), trachea, liver, skin, kidney, heart (e.g., myocardium), bone marrow, mediastinum, joints, and large intestine. Fibrosis may be associated with any disease or condition. Non-limiting examples of diseases or conditions associated with fibrosis include: pulmonary fibrosis (e.g., idiopathic), radiation fibrosis, scleroderma, cystic fibrosis, diabetic nephropathy, hypertensive nephrosclerosis, allograft nephropathy, hepatitis, biliary disease, immune injury, cirrhosis, bridging fibrosis, glial scar, arterial stiffness, arthrofibrosis, Crohn's disease, chronic kidney disease, Dupuytren's contracture, keloid, progressive massive fibrosis, retroperitoneal fibrosis, and adhesive capsulitis.

### Mouse Models

In some embodiments, an animal model of fibrosis is a mouse. A mouse model of the present disclosure may be any immunodeficient mouse known in the art. An immunodeficient mouse is a mouse with an impaired endogenous (e.g., mouse) immune system compared to a non-immunodeficient mouse. An impaired endogenous immune system may be 10%-99%, 15%-95%, 20%-90%, 30%-80%, 40%-70%, or 50%-60% deficient compared to a non-immunodeficient mouse. An impaired endogenous immune system may be 10%, 15%, 20%, 25%, 30%, 35%, 40%, 45%, 50%, 55%, 60%, 65%, 70%, 75%, 80%, 85%, 90%, 95%, or 99% deficient compared to a non-immunodeficient mouse.

An impaired immune system may be measured any method known in the art including, but not limited to: production of mature immune cells (e.g., B cells, T cells, dendritic cells, macrophages, natural killer cells), deficient endogenous cytokine signaling, limited resistance to infection, and reduced survival. In some embodiments, an immunodeficient mouse lacks mature mouse T cells, lacks mature mouse B cells, lacks functional natural killer cells, and is deficient in endogenous (e.g., mouse) cytokine signaling. Mature T cells develop in the thymus and are released to other tissues, including blood, spleen, and lymphatic system. Mature B cells express pathogen-specific antibodies on their surface. Functional natural killer cells recognize and kill malignant and virally transformed cells without previously being exposed. Endogenous (e.g., mouse) cytokine signaling is important in maintaining homeostasis and relies on cytokines to regulate immune, nervous, and endocrine system function. Deficient endogenous (e.g., mouse) cytokine signaling means that the level of cytokine signaling is not sufficient to maintain immune system homeostasis compared to an endogenous immune system that is not deficient. Lack of mature cells (e.g., T cells or B cells), functional cells, (e.g., natural killer cells), deficient cytokine signaling, or some combination thereof may be a 10-99%, 5%-95%, 20%-90%, 30%-80%, 40%-70%, or 50%-60% decrease compared to a non-immunodeficient mouse. Lack of mature cells (e.g., T cells or B cells), functional cells (e.g., natural killer cells), deficient cytokine signaling, or some combination thereof may be a 10%, 15%, 20%, 25%, 30%, 35%, 40%, 45%, 50%, 55%, 60%, 65%, 70%, 75%, 80%, 85%, 90%, 95%, or 99% decrease compared to a non-immunodeficient mouse.

Lack of mature cells or functional cells (e.g., T cells, B cells, NK cells) may be assessed by any method known in the art including, but not limited to: flow cytometry; quantitative PCR (qPCR) of T cell markers (e.g., CD3, CD8, CD4, CD25, CD127, CD152), B cells markers (e.g., CD19, IgM, BCAP), and NK cells (e.g., CD224, CD122, NK11, NKp46, Ly49, CD11b, CD49b); immunofluorescence, and ELISA. Deficient cytokine signaling (e.g., mouse cytokine signaling) may be assessed by any method known in the art including, but not limited to: flow cytometry, qPCR of cytokines (e.g., IL-2, IL-7, IL-15, IFNγ, IL-4, IL-5, IL-9, IL-13, IL-25, IL-17A, IL-17F, IL-22, TNFα, IL-12, CCL3, GM-CSF, IL-6, IL-10, TGFβ, IL18, IL-21), immunofluorescence, and ELISA.

Immunodeficient mice by created by any method known in the art including, but not limited to: genomic modification (e.g., transgenic modification), drug administration (e.g., dexamethasone, cyclophosphamide), and irradiation.

An immunodeficient mouse may express any human cytokine or combination of human cytokines that increases the efficacy of the immunodeficient mouse as an animal model (e.g., of human fibrosis). A cytokine is a protein or peptide that modulates the activities of individual cells or tissues (e.g., other human cells, mouse cells). Non-limiting examples of types of human cytokines that may be expressed in a human fibrosis model include: hematopoietic cytokines, lymphokines, monokines, interferons, and chemokines.

In some embodiments, an immunodeficient mouse expresses a human hematopoietic cytokine. Human hematopoietic cytokines are extracellular proteins and peptides that stimulate hematopoietic cells (e.g., hematopoietic stem cells) to develop into differentiated blood cells (e.g., neutrophils, basophils, eosinophils, macrophage). Non-limiting examples of human hematopoietic cytokines include: interleukin 3 (IL-3), granulocyte/macrophage colony stimulating factor (GM-CSF), macrophage colony stimulating factor (M-CSF), thrombopoietin (TPO), IL-11, erythropoietin (EPO), granulocyte colony stimulating factor (G-CSF), IL-5, IL-6, IL-2, IL-7, IL-4, IL-17, and IL-15.

In some embodiments, an immunodeficient mouse described herein expresses 1-20, 2-19, 3-18, 4-17, 5-16, 6-15, 7-14, 8-13, 9-12, or 10-11 human cytokines (e.g., human hematopoietic cytokines). In some embodiments, an immunodeficient mouse described herein expresses 1, 2, 3, 4, 5, 6, 7, 8, 9, 10, 11, 12, 13, 14, 15, 16, 17, 18, 19, 20, or more human cytokines. In some embodiments, a human cytokine expressed in an immunodeficient mouse is involved in macrophage development, proliferation, or a combination thereof. Non-limiting examples of human cytokines CSF1, interleukin 34 (IL-34), GM-CSF (CSF2), Haem, tumor growth factor R (TGFβ), IL-10, and LTα1β2.

A human cytokine may be expressed in an immunodeficient mouse by any method known in the art including, but not limited to: from a transgene in immunodeficient mouse and from a virus (e.g., lentivirus, adenovirus, adeno-associated virus) comprising a sequence encoding a human cytokine. A transgene is a gene that is transferred from one organism (e.g., human) to another (e.g., mouse). In some embodiments, a human cytokine is expressed from a transgene. A transgene in an immunodeficient mouse may be inserted into the immunodeficient mouse's genome or encoded in a vector that expresses the transgene.

An immunodeficient mouse may be any immunodeficient mouse known in the art including, but not limited to: a non-obese diabetic (NOD; e.g., NSG™, NOG, NRG) mouse, a severe combined immunocompromised (SCID) mouse (e.g., BALB/cA-scid, B6-scid, AKR-scid, a nude mouse (e.g., CBA/N-nu, B6-nu, C3H-nu, ICR-nu), a BALB/c mouse (e.g., BALB/cA-Rag2^{null}I2rγ^{null} (BRG)), or a C57BL/6 immunocompromised mouse (e.g., C57BL/6 Rag2^{null}I2rγ^{null} (B6RG) (see, e.g., Ito et al., (2012) Cellular & Molecular Immunology, 9: 208-214).

In some embodiments, an immunodeficient mouse is a NOD SCID gamma (NSG™) mouse (e.g., JAX Stock No.: 005557). The NSG™ mouse is an immunodeficient mouse that lacks mature T cells, B cells, and natural killer (NK) cells, is deficient in multiple cytokine signaling pathways, and has many defects in innate immunity (see, e.g., (Shultz, Ishikawa, & Greiner, 2007; Shultz et al., 2005; Shultz et al., 1995), each of which is incorporated herein by reference). The NSG™ mouse, derived from the non-obese diabetic (NOD) mouse strain NOD/ShiLtJ (see, e.g., (Makino et al., 1980), which is incorporated herein by reference), includes an inactivated Prkdc^{scid} allele (also referred to as the “severe combined immunodeficiency” mutation or the “scid” mutation) and an inactivated Il2rg^{tm1Wjl} allele. The Prkdc^{scid} mutation is a loss-of-function mutation in the mouse homolog of the human PRKDC gene—this mutation essentially eliminates adaptive immunity (see, e.g., (Blunt et al., 1995; Greiner, Hesselton, & Shultz, 1998), each of which is incorporated herein by reference). The Il2rg^{tm1Wjl} mutation is a null mutation in the gene encoding the interleukin 2 receptor gamma chain (IL2Rγ, homologous to IL2RG in humans), which blocks NK cell differentiation, thereby removing an obstacle that prevents the efficient engraftment of primary human cells ((Cao et al., 1995; Greiner et al., 1998; Shultz et al., 2005), each of which is incorporated herein by reference). A loss-of-function mutation, as is known in the art, results in a gene product with little or no function. By comparison, a null mutation results in a gene product with no function. An inactivated allele may be a loss-of-function allele or a null allele.

In some embodiments, an immunodeficient mouse is an NSG-SGM3 mouse (e.g., JAX Stock No.: 013062). An NSG-SGM3 mouse has the genotype NOD.Cg-Prkdc^{scid} Il2rg^{tm1Wjl} Tg(CMV-IL3,CSF2,KITLG)1Eav/MloySzJ. NSG-SGM3 mice express a human interleukin 3 gene (IL3; Gene ID: 3562), a human granulocyte/macrophage-stimulating factor gene (CSF2 gene; GM-CSF; Gene ID: 1437), and a human Steel factor gene (KITLG, SCF, SF; Gene ID: 4254), each expressed from a human cytomegalovirus (CMV) promoter.

In some embodiments, an immunodeficient mouse is an NSG-CSF1 mouse (e.g., JAX Stock No.: 028654). An NSG-CSF1 mouse has the genotype NOD.Cg-Prkdc^{scid}Il2rg^{tm1wjl} Tg(CSF1)3Sz/SzJ. NSG-SCF1 mice express a human colony stimulating factor 1 gene (CSF1; Gene ID: 1435) expressed from an endogenous human CSF1 promoter.

In some embodiments, an immunodeficient mouse is a modified NSG™ model known in the art. Non-limiting examples of modified NSG™ models include: NRG (NOD.Cg-Rag1^{tm1Mom}Il2rg^{tm1wjl}/SzJ; e.g., JAX Stock No.: 007799), NSG-HLA-A2.1 (NOD.Cg-Mcph1^{Tg(HLA-A2.1)1Enge}Prkdc^{scid}Il2rg^{tm1Wjl}/SzJ; e.g., JAX Stock No.: 009617), NSG-HLA-A2/HHD (NOD.Cg-Prkdc^{scid} I2rg^{tm1Wjl} Tg(HLA-A/H2-D/B2M)1Dvs/SzJ; e.g., JAX Stock No.: 014570), NSG-DR1 (NOD.Cg-Tg(HLA-DRA*0101,HLA-DRB1*0101) 1Dmz Prkdc^{scid} Il2rg^{tm1Wjl}/GckRolyJ; e.g., JAX Stock No.: 012479), NSG-DR4 (NOD.Cg-Prkdc^{scid} Il2rg^{tm1Wjl} H2-Ab1^{tm1Doi} Tg(HLA-DRB1)31Dmz/SzJ; e.g., JAX Stock No.: 017637), NSG-B2M (NOD.Cg-B2m^{tm1Unc} Prkdc^{scid} Il2rg^{tm1}Wjl/SzJ; e.g., JAX Stock No.: 010636), NSG-(K^{b}D^{b})^{null} (NOD.Cg-Prkdc^{scid} H2-K1^{tm1Bpe} H2-D1^{tm1Bpe} Il2rg^{tm1Wjl}/SzJ; e.g., JAX Stock No.: 023848), NSG-MHC I/II KO (NOD.Cg-Prkdc^{scid} H2-Ab1^{em1Mvw} H2-K1^{tm1Bpe} H2-D1^{tm1Bpe} Il2rg^{tm1Wjl}/SzJ; e.g., JAX Stock No.: 025216), NSG-IL15 (NOD.Cg-Prkdc^{scid} Il2rg^{tm1Wjl} Tg(IL15)1Sz/SzJ; e.g., JAX Stock No.: 030890), NBSGW (NOD.Cg.Kit^{W-41J}Tyr⁺Prkdc^{scid}Il2rg^{tm1Wjl}/ThomJ; e.g., JAX Stock No.: 026622), NSG-PiZ (NOD.Cg-Prkdc^{scid}Il2rg^{tm1Wjl}Tg(SERPINA1*E342K) #Slcw/SzJ; e.g., JAX Stock No.: 028842), and NSG-Tlr4 KO (NOD.Cg-Tlr4^{lps-del} PrkdC^{scid}Il2rg^{tm1Wjl}/SzJ; e.g., JAX Stock No.: 033704).

### Humanization

A human fibrosis model of the present disclosure is a humanized model (e.g., humanized immunodeficient mouse). Humanization refers to engraftment with human immune cells such that the fibrosis model develops a human immune system. In some embodiments, a humanized mouse is an immunodeficient mouse (e.g., NSG™, NSG-SGM) engrafted with human immune cells.

A humanized fibrosis model (e.g., mouse model) need not develop every type of human immune cell. Non-limiting examples of human immune cells that may be developed in a model of the present disclosure include: macrophages, myeloid progenitor cells, lymphoid progenitor cells, megakaryocytes, platelets, erythrocytes, myeloblasts, basophils, eosinophils, neutrophils, mast cells, monoblasts, monocytes, dendritic cells, plasma cells, precursor T-cells, killer T-cells, memory T-cells, B cells, memory B cells, and natural killer cells. In some embodiments, a humanized fibrosis model (e.g., mouse model) develops 1-22, 2-21, 3-20, 4-19, 5-18, 6-17, 7-16, 8-15, 9-14, 8-13, 9-12, or 10-11 types of human immune cells. In some embodiments, a humanized fibrosis model develops 1, 2, 3, 4, 5, 6, 7, 8, 9, 10, 11, 12, 13, 14, 15, 16, 17, 18, 19, 20, 21, or 22 types of human immune cells.

In some embodiments, an immunodeficient mouse supports engraftment of myeloid lineage cells. Supporting engraftment means that myeloid lineage cells may be implanted into and proliferate in an immunodeficient mouse to produce a humanized mouse that mimics a human immune system. Myeloid lineage cells are blood cells that arise from bone marrow. Myeloid lineage cells are derived from a hematopoietic stem cell and differentiate into myeloid progenitor cells, megakaryocytes, platelets, eosinophils, basophils, erythrocytes, monocytes, dendritic cells, macrophages, and neutrophils. In some embodiments, an immunodeficient mouse supports engraftment of CD33+ human myeloid lineage cells. CD33 is a transmembrane receptor protein expressed on the surface of and can used to distinguish myeloid cells from lymphoid cells. CD33 is expressed on the surface of myeloid progenitor cells, which are derived from hematopoietic stem cells and develop into differentiated cells including, but not limited to: megakaryocytes (CD41a+, CD42b+), thrombocytes (CD41a+, CD42b+), erythrocytes (CD36+, CD47+, CD71+), mast cells (CD117+, CD123+, CD203c+, CD32+, CD33+, CD45+), basophils (CD117+CD123+, CD164+, CD194+, CD24+. CD294+, CD54+, CD63+, CD69+), neutrophils (CD13+, CD14+, CD141+, CD15+. CD16+, CD183+, CD32+, CD33+, CD44+, CD63+, CD66+, CD68+), eosinophils (CD193+, CD198+, CD23+, CD294+, CD32+. CD45+, CD62L+, CD88+, CD9), monocytes (CD115+, CD118+, CD14+, CD16+, CD192+, CD2+, CD31+, CD56+, CD62L+), macrophages (CD68+, CSF1R, EMR1, CD105+, CD14+, CD15+, CD163+, CD195+, CD282+, CD284+, CD33+, CD64+, CD68+, CD80+), and dendritic cells (CD197+. CD205+, CD207+, CD209+, CD273+, CD304+, CD4+, CD40+. CD80+, CD83+, CD86+).

In some embodiments, an immunodeficient humanized mouse contains human innate immune cells. The human innate immune system generates a rapid (e.g., compared to the adaptive immune system), non-specific inflammatory response to infectious agents (e.g., bacteria, virus, foreign matter). Innate immune cells are the body's generalized first line of defense against immunogens and are important in controlling infections during the first 7 days after infection. Non-limiting examples of innate immune cells include: myeloid progenitor cells, megakaryocytes, platelets, eosinophils, basophils, erythrocytes, monocytes, dendritic cells, macrophages, and neutrophils.

In some embodiments, an immunodeficient humanized mouse contains human innate myeloid cells. Innate myeloid cells are a type of innate immune cells derived from bone marrow and include myeloid progenitor cells, megakaryocytes, platelets, eosinophils, basophils, erythrocytes, monocytes, dendritic cells, macrophages, and neutrophils. Innate myeloid cells may be identified by any method known in the art including, but not limited to: assaying for expression of cell-specific markers and brightfield microscopy. In some embodiments, innate myeloid cells may be identified by assaying for expression of cell-specific markers. The assaying may be done by any method known in the art including, but not limited to: fluorescence activated cell sorting (FACS), immunofluorescence, and immunohistochemistry. The cell-specific markers may be any marker known in the art to distinguish myeloid cells (or specific myeloid cells) from other immune cells. In some embodiments, the innate myeloid cells express CD45, CD33, or a combination thereof. In some embodiments, the innate myeloid cells are macrophages and express CD68, CSF1R, EMR1, or some combination thereof.

In some embodiments, an immunodeficient humanized mouse contains human adaptive immune cells. The human adaptive immune system is a slower (e.g., compared to innate immune system), specific inflammatory response to infections agents. Adaptive immune cells are the body's specialized second line of defense and include lymphoid progenitor cells, T cells, B cells, plasma cells, and natural killer cells. T cells and B cells are the main components of the adaptive immune system. T cells bind an antigen on the infectious, recognize the antigen, kill the infectious agent, and mount a systemic immune response to fight the infection. B cells bind and produce antibodies to a specific antigen for subsequent infection.

Human immune cells for humanizing a model (e.g., an immunodeficient mouse) may be any source of human immune cells known in the art. Non-limiting examples of sources of human immune cells include: fetal tissue, hematopoietic stem cells, peripheral blood mononuclear cells, human umbilical cord blood cells, induced pluripotent stem cells (iPSCs), and any combination thereof.

In some embodiments, an immunodeficient mouse is humanized by engrafting with human fetal tissue. Engrafting an immunodeficient mouse with human fetal tissue is described in Shultz et al., (2013), Nat. Rev. Immunol., which is incorporated by reference herein. Briefly, engrafting an immunodeficient mouse with human fetal tissue means that human fetal tissue is implanted into and proliferates in an immunodeficient mouse. Engrafting an immunodeficient mouse with human fetal tissue may allow the immunodeficient mouse to model the engrafted tissue's behavior. For example, when an immunodeficient mouse is engrafted with human fetal immune tissue, the immunodeficient mouse may model human immune system behavior. Human fetal tissue may include any fetal tissue. Non-limiting examples of fetal tissues that may be engrafted into an immunodeficient mouse for humanization include: bone marrow, liver, thymus, spleen, lymph nodes, lymph vessels, skin, large intestine, small intestine, pancreas, liver, brain, stomach, kidney, heart, and lung. In some embodiments, an immunodeficient mouse is engrafted with human fetal immune tissue. In some embodiments human fetal immune tissue includes bone marrow, liver, thymus, spleen, lymph nodes, lymph vessels, skin, or some combination thereof. In some embodiments, an immunodeficient mouse is engrafted with human fetal bone marrow, liver, and thymus (BLT) tissues.

In some embodiments, the immunodeficient mouse is engrafted with human hematopoietic stem cells. Human hematopoietic stem cells are human cells that can differentiate into and produce human blood cells indefinitely. Engrafting an immunodeficient mouse with human hematopoietic stem cells is described in Aryee et al., (2014), Methods Mol. Biol., which is incorporated by reference herein. Briefly, engrafting an immunodeficient mouse with human hematopoietic stem cells means that human hematopoietic stem cells are implanted into and proliferate in an immunodeficient mouse. Human hematopoietic stem cells develop into differentiated cells that can no longer reproduce indefinitely and are committed to a specific cell lineage as a result of cell signaling (e.g., cytokines). Human hematopoietic stem cells may be identified by any method known in the art including, but not limited to: detecting expression of a cell surface antigen specific to human hematopoietic stem cells (e.g., CD45, CD34, CD59, CD90) and lack of expression of a cell surface antigen specific to differentiated cells (e.g., CD33, CD41a, CD42b, CD36, CD47, CD71, CD117, CD123, CD203c, CD32, CD33, CD45).

Multiple human tissues may be engrafted into an immunodeficient mouse. This may be done for numerous reasons, including, but not limited to: more effective modeling of a human system if the multiple human tissues are part of the same system or modeling of different human tissue systems. Multiple human tissues that are engrafted into an immunodeficient mouse may be autologous or allogenic. Autologous means that the multiple human tissues that are engrafted into an immunodeficient mouse are derived from the same human. Allogenic means that the multiple human tissues that are engrafted into an immunodeficient mouse are derived from different humans. In some embodiments, multiple human tissues that are engrafted into an immunodeficient mouse are autologous.

Human cells (e.g., fetal tissue, hematopoietic stem cells) may be engrafted into a model (e.g., an immunodeficient mouse model of fibrosis) by any method known in the art. Non-limiting examples of methods of engrafting human immune cells into a model include: injecting human immune cells (e.g., intravenously, intramuscularly, intraarterially) and implanting human immune cells (e.g., renal subscapular space, epididymal fat pad, peritoneal cavity). In some embodiments, an immunodeficient mouse is humanized with fetal bone marrow, liver, and thymus tissues and hematopoietic stem cells.

### Implanting Exogenous Biomaterial

The present disclosure provides a model of human fibrosis. The model of human fibrosis is produced, in some embodiments, by implanting an exogenous biomaterial into a model (e.g., a humanized immunodeficient mouse). In some aspects of the present disclosure, an exogenous biomaterial is implanted into a mouse model. An exogenous biomaterial is a natural or synthetic material that does not originate in the model but that may be used in treating disease.

An exogenous biomaterial may be implanted by any method known in the art including, but not limited to: surgical incision, injection, ingestion, and inhalation. In some embodiments, the exogenous biomaterial is implanted by surgical incision. In some embodiments, the exogenous biomaterial is implanted by injection. An exogenous biomaterial may be implanted in any location in a model of human fibrosis (e.g., a humanized immunodeficient mouse). Non-limited examples of locations to implant an exogenous biomaterial include: peritoneal cavity, subcutaneous space, thoracic cavity, abdominal cavity, renal subscapular space, joints (e.g., knee, hip, shoulder, fingers), trachea, heart, blood vessels, gastrointestinal tissues, face, and bones.

In some embodiments, an exogenous biomaterial is a natural exogenous biomaterial. A natural exogenous biomaterial is a substance that can be found in nature and is known to be or thought to be useful in treating disease. Non-limiting examples of natural exogenous biomaterials include: alginate, agarose, cellulose, starch, silk fibroin, chitosan, fibrin, gelatin, collagen, hyaluronic acid. In some embodiments, an exogenous biomaterial includes a combination of natural exogenous biomaterials. In some embodiments, an exogenous biomaterial includes 1-10, 2-9, 3-8, 4-7, or 5-6 natural exogenous biomaterials. In some embodiments, an exogenous biomaterial includes 1, 2, 3, 4, 5, 6, 7, 8, 9, or 10 or more natural exogenous biomaterials.

In some embodiments, an exogenous biomaterial is a synthetic exogenous biomaterial. A synthetic exogenous biomaterial is a substance that is not found in nature and is known to be or thought to be useful in treating disease. Non-limiting examples of a synthetic exogenous biomaterials include: metals (e.g., stainless steel, cobalt, chromium, titanium), ceramics (e.g., aluminum oxide, zirconia, calcium phosphates), glass, and polymers (e.g., polystyrene, polydimethylsiloxane (PDMS), polylactic acid (PLA), polycaprolactone (PCL), polyoxymethylene (POM), P(HEMA), PEUU, PGA, PLGA, PC-BU, Polyester-urethane, PLGA, PLLA/PCL, polyamide (PA), polyethylene glycol (PEG)). In some embodiments, an exogenous biomaterial includes 1-10, 2-9, 3-8, 4-7, or 5-6 synthetic exogenous biomaterials. In some embodiments, an exogenous biomaterial includes 1, 2, 3, 4, 5, 6, 7, 8, 9, or 10 or more synthetic exogenous biomaterials.

In some embodiments, an exogenous biomaterial is a biomedical device. A biomedical device is an instrument, apparatus, implement, machine, contrivance, implant, or in vitro reagent used: in the detection of a disease or a condition, in the cure, mitigation, treatment, or prevention of a disease, or to affect the structure or function of the body of a human or other animal. A biomedical device may be a natural exogenous biomaterial, a synthetic exogenous biomaterial, or a combination thereof. Non-limiting examples of biomedical devices include: cardiac pacemakers, cardiac defibrillators, coronary stents, joint implants (e.g., hip, should, knee), interocular lenses, insulin pumps, surgical pins, surgical rods, and surgical screws.

In some embodiments, an exogenous biomaterial includes a combination of natural exogenous biomaterials and synthetic exogenous biomaterials. In some embodiments, the exogenous biomaterial includes 1-10, 2-9, 3-8, 4-7, or 5-6 natural exogenous biomaterials and synthetic exogenous biomaterials. In some embodiments, an exogenous biomaterial includes 1, 2, 3, 4, 5, 6, 7, 8, 9, or 10 or more natural exogenous biomaterials and synthetic exogenous biomaterials.

Exogenous biomaterials may be used for any purpose known in the art. Non-limiting examples of exogenous biomaterials applications include: orthopedics (e.g., joint replacements), bone stabilization (e.g., screws, plates, pins, bone cement), sensory correction (e.g., intraocular lenses, cochlear implant, contact lenses), artificial ligaments and tendons, dental implants, cardiovascular applications (e.g., blood vessel prostheses, heart valves, pacemakers, vascular grafts, stents), wound healing (e.g., skin repair devices), breast implants, drug delivery mechanisms (e.g., nanoparticles, medical pumps), nerve conduits, surgical sutures, surgical clips, surgical staples, and surgical mesh.

### Methods of Use

The present disclosure provides, in some aspects, methods of assessing human fibrosis associated with an exogenous biomaterial in a model (e.g., a humanized immunodeficient mouse model). Fibrosis associated with an exogenous biomaterial is fibrosis that occurs after an exogenous biomaterial is implanted into a model. Human fibrosis associated with an exogenous biomaterial may be fibrosis that is completely (100%) attributable to the exogenous biomaterial or fibrosis that is partially (0.1%-99.9%) attributable to the exogenous biomaterial.

Human fibrosis associated with an exogenous biomaterial may be assessed in a model (e.g., a humanized immunodeficient mouse model) at any time after the exogenous biomaterial has been implanted. Assessing fibrosis (e.g., human fibrosis) means monitoring or measuring the development or progression of fibrosis associated with an exogenous biomaterial in a model.

Human fibrosis may be assessed in any biological sample from a model by any method known in the art. Non-limiting examples of biological samples include: solid tissue samples (e.g., peritoneal tissue, skin tissue, lung tissue) and fluid samples (e.g., whole blood, serum, plasma). Multiple biological samples may be assessed simultaneously and need not have been collected at the same time or be the sample type of biological sample. Non-limiting methods of assessing human fibrosis include: measuring gene expression, measuring protein expression, quantifying cell immune staining, visualizing cell morphology, measuring metabolite concentration, and imaging tissues in vivo (e.g., X-ray, magnetic resonance imaging (MRI), transient elastography (TE), acoustic radiation force impulses (ARFI)).

In some embodiments, assessing human fibrosis in a model (e.g., a humanized immunodeficient mouse) is measuring gene expression. Gene expression may be measured by any method known in the art including, but not limited to: quantitative polymerase chain reactions (qPCR), quantitative real time PCT (qRT-PCR), enzyme-linked immunosorbent assay (ELISA), Western blot, serial analysis of gene expression (SAGE), and Northern blot.

In some embodiments, assessing human fibrosis in a model is measuring protein expression. Protein expression may be measured by any method known in the art including, but not limited to, Proteins upregulated include, but are not limited to: Western blot, ELISA, and fluorescence activated cell sorting (FACS).

Genes and proteins upregulated by fibrosis include, but are not limited to: colony stimulating factor 1 (CSF1), colony stimulating factor 2 (CSF2, GM-CSF), transforming growth factor beta (TGFβ), cluster of differentiation (CD) 68, CSF1 receptor (CSF1R), EGF-like module-containing mucin-like hormone receptor-like 1 (EMR1), CD19, interferon gamma receptor 1 (IFNGR1), C—X—C chemokine receptor 4 (CXCR4), CD4, chemokine ligand 3 (CCL13), lysozyme (LYZ), interleukin 6 receptor (IL6R), CCL24, progranulin (GRN), IL-13 receptor, alpha 1 (IL13RA1), IL-2 receptor alpha (IL2RA), inhibin subunit beta A (INHBA), CCL3, SP1 transcription factor (SP1), integrin subunit alpha L (ITGAL), C—X—C chemokine ligand 3 (CXCL3), CXCL8, CCL22, high mobility group box 1 (HMGB1), IL1DRB, carboxypeptidase A3 (CPA3), TNF receptor superfamily member 14 (TNFRSF14), norrin (NDP), integrin alpha M (ITGAM), CCR1, lymphocyte antigen 96 (LY96), nicotinamide phosphoribosyltransferase (NAMPT), TNFRSF18, atypical chemokine receptor 2 (ACKR2), ACKR3, amphiregulin (AREG), CCL1G, CCL8, CD19, CD209, chemokine like receptor 1 (CMKLR1), CKLF Like MARVEL transmembrane domain containing 7 (CMTM7), CMTM8, CX3CL1, CXCL10, CXCL13, CXCL9, FOXP3, IL12RB2, IL15, IL17RB, IL17RC, IL18R1, ILlA, IL3RA, interleukin 6 cytokine family signal transducer (IL6ST), tyrosine protein kinase (KIT), killer cell lectin like receptor K1 (KLRK1), lymphotoxin beta receptor (LTBR), phosphatidylinositol glycan anchor biosynthesis class Q (PIGQ), pro-platelet basic protein (PPBP), CD46, thrombospondin 1 (THBS1), thrombopoietin (THPO), TNF receptor superfamily member 10a (TNFRSF10A), TNFRSF10B, TNFRSF10C, TNFRSF10D, TNFRSF13C, TNFRSF25, and TNFSF13B. Genes and proteins downregulated by fibrosis include, but are not limited to: FAS (Fas Cell Surface Death Receptor), toll-like receptor 2 (TLR2), carboxypeptidase A3 (CPA3), interferon alpha and beta receptor subunit 2 (IFNAR2), and cardiotrophin 1 (CTF1).

In some embodiments, assessing human fibrosis in a model (e.g., humanized immunodeficient mouse) is quantifying cell immune staining. Cell immune staining is labeling proteins on or in cells with an antibody conjugated to a detectable label and detecting the label. Cell immune staining is quantified by measuring the signal from the detectable label. A detectable label may be any label known in the art including, but not limited to: fluorescent labels, metal labels, and biotin labels. In some embodiments, a detectable label on an antibody is a fluorescent label. Non-limiting examples of fluorescent labels include: Cy2, Cy3, Cy3B, Cy3.5, Cy5, Cy5.5, Cy7, CyPet, TRITC, X-Rhodamine, Lissamine Rhodamine B, Texas Red, Allophycocyanin, Cy7, hydroxycoumarin, aminocoumarin, methoxycoumarin, Cascade Blue, Pacific Blue, Pacific Orange, Lucifer yellow, NBD, R-Phycoerythrin, Red 613, PerCP, TruRed, FluorX, Fluorescein, BODIPY-FL, G-Dye100, G-Dye200, G-Dye300, G-Dye-400, Sapphire, Cerulean, mCFP, mTurquoise2, ECFP, Emerald, Azami Green, ZsGreenl, EYFP, YFP, GFP, RFP, CFP, tdTomato, Citrine, and mCitrine.

In some embodiments, cell immune staining is performed using fluorescent labels. In some embodiments, cell immune staining may be used to quantify the number of cells positive for a particular cell protein associated with fibrosis (e.g., CD45+ myeloid progenitor cells, CD33+ myeloid cells, CD68+ macrophages). This number of cells positive for a particular marker associated with fibrosis may be used to indicate the proliferation and survival of these cells, and the progression of fibrosis.

Quantifying cell immune staining and protein expression may be combined to analyze fibrosis migrating from the exogenous biomaterial implant site into surrounding tissues. Briefly, cell proteins that identify specific cells (CD45+ myeloid progenitor cells, CD33+ myeloid cells, CD68+ macrophages) may be stained with an antibody conjugated to a detectable label (e.g., fluorescent antibody), signal from the detectable label may be quantified as a measure of expression of that protein, and the location of the labeled protein in relation to the implant site may be mapped. The greater the protein expression and movement of cells associated with human fibrosis (e.g., CD68+ macrophages) into tissue surrounding the exogenous biomaterial implant site, the greater the likelihood that fibrosis is progressing.

In some embodiments, human fibrosis is assessed at an early stage after exogenous biomaterial implantation (e.g., 0 hours, 1 hour, 2 hours, 4 hours, 12 hours, 24 hours, 2 days, 3 days, 4 days, 5 days, 6 days, 1 week) to gain a baseline of human fibrosis. In some embodiments, human fibrosis is assessed at an intermediate stage after exogenous biomaterial implantation (e.g., 8 days, 9 days, 10 days, 11 days, 12 days, 13 days). In some embodiments, human fibrosis is assessed at a late stage after exogenous biomaterial implantation (e.g., 2 weeks-3 weeks, 22 days-4 weeks, 5 weeks-10 weeks, 71 days-26 weeks, 183 days-52 weeks, or longer).

Human fibrosis may be assessed multiple times after exogenous biomaterial implantation. In some embodiments, human fibrosis may be assessed 1-30, 2-29, 3-28, 4-27, 5-26, 6-25, 7-24, 8-23, 9-22, 10-21, 11-20, 12-19, 13-18, 14-17, or 15-16 times. In some embodiments, human fibrosis may be assessed 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more times. In some embodiments when human fibrosis is assessed multiple times after exogenous biomaterial implantation, the multiple times are in the same stage (e.g., early, intermediate, late). In some embodiments when human fibrosis is assessed multiple times after exogenous biomaterial implantation, the multiple times are in different stages (e.g., early, intermediate, late, or a combination thereof).

In one aspect, a human fibrosis model provided herein is used in a method to predict fibrosis associated with an exogenous biomaterial. Predicting fibrosis associated with an exogenous biomaterial includes: obtaining one or more biological samples from a human fibrosis model (e.g., a humanized immunodeficient mouse) at an early stage after an exogenous biomaterial is implanted; measuring gene expression, protein expression, and/or cell immune staining of genes, proteins, and cell types associated with fibrosis (e.g., CD45+ myeloid progenitor cells, CD33+ myeloid progenitor cells, CD68+ macrophages); and predicting fibrosis based on known gene expression, protein expression, and/or cell immune staining results associated with fibrosis. For example, the presence of human macrophages (CD68+, CSFR1+, EMR1+) at an early stage after exogenous biomaterial implantation may indicate that fibrosis is developing or will develop.

In a further aspect, a human fibrosis model provided herein is used in a method to assess a putative fibrosis treatment. A putative fibrosis treatment may be any drug or compound known in the art to or suspected to aid in the treatment of fibrosis. Treatment of fibrosis may be a decrease in symptoms of or cells associated with fibrosis. A putative treatment of fibrosis may decrease symptoms or cells associated with fibrosis by 10-99%, 5%-95%, 20%-90%, 30%-80%, 40%-70%, or 50%-60% compared to non-treated models of human fibrosis. In some embodiments, a putative treatment of fibrosis may decrease symptoms of or cells associated with fibrosis by 10%, 15%, 20%, 25%, 30%, 35%, 40%, 45%, 50%, 55%, 60%, 65%, 70%, 75%, 80%, 85%, 90%, 95%, or 99% compared to non-treated models of human fibrosis. Non-limiting examples of putative treatments of human fibrosis include: pirfenidone (Esbiret®), nintedanib (Ofev®), benazepril, vitamin E, IL-4/IL-13 dual antibody, Lisinopril, ramipril, a-Tocopherol, interferon-alpha, PPAR-alpha agonist, and matrix metalloproteinase inhibitors.

A putative fibrosis treatment may be administered at any appropriate dosage. An appropriate dosage will depend on numerous factors including, but not limited to: age, weight, disease state, other treatments being administered, other treatments previously administered. A putative fibrosis treatment may be administered at 0.1 μg/kg-1,000 mg/kg, 1 μg/kg-100 mg/kg, 10 μg/kg-10 mg/kg, or 100 μg/kg-1 mg/kg. In some embodiments, a putative fibrosis treatment is administered at 0.1 μg/kg, 1.0 μg/kg, 10 μg/kg, 100 μg/kg, 1 mg/kg, 10 mg/kg, 100 mg/kg, or 1,000 mg/kg or more. A putative fibrosis treatment may be administered by any method known in the art. Non-limiting methods of administered a putative fibrosis treatment include: injection (e.g., intravenous, intramuscular, intraarterial), inhalation, ingestion, and engraftment.

In a further aspect, a humanized mouse model provided herein may be used as a surrogate pre-clinical diagnostic tool for screening exogenous biomaterial immunogenicity. Immunogenicity is a human immune response in a humanized mouse model caused by the exogenous biomaterial. Thus, an experimental exogenous biomaterial(s) may be implanted into a humanized mouse model(s) provided herein and the humanized mouse model may be assessed for immunogenicity to the exogenous biomaterial over time. When there is a no or minor immunogenicity in response to an exogenous biomaterial being implanted in a humanized mouse model, the exogenous biomaterial is a candidate for human use. When there is intermediate or major immunogenicity in response to an exogenous biomaterial being implanted in a humanized mouse model, the exogenous biomaterial may not be a candidate for human use. Minor immunogenicity is an increase of 1-25% over a control, intermediate immunogenicity is an increase of 25%-50% over a control, and major immunogenicity is an increase of 50%-100% or more over a control. A control may be a humanized mouse model that does not have an exogenous biomaterial implanted or a humanized mouse model that has an exogenous biomaterial implanted that is known to not be immunogenic.

Immunogenicity may be assessed by any method known in the art. Non-limiting examples of assessing immunogenicity include: measuring human immune cell proliferation, measuring human immune cell growth, and assessing human immune cell signaling as a result of contact with the exogenous biomaterial. A control for assessing immunogenicity may be a humanized mouse model that does not have an exogenous biomaterial implanted or a humanized mouse model that has an exogenous biomaterial implanted that is known to not be immunogenic.

Immunogenicity may involve any human immune cell known in the art. Non-limiting examples of human immune cells that may be involved in an immunogenic response include: megakaryocytes, thrombocytes, erythrocytes, mast cells, basophils, neutrophils, eosinophils, monocytes, macrophages, dendritic cells, T cells, B cells, plasma cells, natural killer cells, or any combination thereof. In some embodiments, immunogenicity involves 1-10, 2-9, 3-8, 4-7, or 5-6 human immune cells. In some embodiments, immunogenicity includes 1, 2, 3, 4, 5, 6, 7, 8, 9, or 10 or more human immune cells.

In some embodiments, implanting an exogenous biomaterial stimulates immunogenicity by increasing proliferation of human immune cells relative to a control. Proliferation of human immune cells may be measured by any method known in the art, including, but not limited to: a metabolic activity assay (e.g., MTT assay, XTT assay, MTS assay, WST1 assay), a cell proliferation marker assay (e.g., Ki-67 antibody, PCNA antibody, topoisomerase IIB antibody, phosphorylated histone H3), an ATP concentration assay (e.g., luciferase-based assay, radioactivity-based assay), or a DNA synthesis assay (e.g., radiolabeled 3H-thymine, bromodeoxyuridine). In some embodiments, human immune cell proliferation is increased by 5%-100%, 10%-95%, 15%-90%, 20%-85%, 25%-80%, 30%-75%, 35%-70%, 40%-65%, 45%-60%, or 50%-55% or more compared to a human immune cell not contacted with an exogenous biomaterial. In some embodiments, human immune cell proliferation is increased by 5%, 10%, 15%, 20%, 25%, 30%, 35%, 40%, 45%, 50%, 55%, 60%, 65%, 70%, 75%, 80%, 85%, 90%, 95%, or 100% or more compared to a human immune cell not contacted with an exogenous biomaterial.

In some embodiments, implanting an exogenous biomaterial stimulates immunogenicity by increasing growth of human immune cells relative to a control. Growth of human immune cells may measured be any method known in the art, including, but not limited to: microscopy (e.g., immunofluorescence, immunohistochemistry, brightfield microscopy) and staining (H&E, Masson's Trichome). In some embodiments, human immune cell growth is increased by 5%-100%, 10%-95%, 15%-90%, 20%-85%, 25%-80%, 30%-75%, 35%-70%, 40%-65%, 45%-60%, or 50%-55% or more compared to a human immune cell not contacted with an exogenous biomaterial. In some embodiments, human immune cell growth is increased by 5%, 10%, 15%, 20%, 25%, 30%, 35%, 40%, 45%, 50%, 55%, 60%, 65%, 70%, 75%, 80%, 85%, 90%, 95%, or 100% or more compared to a human immune cell not contacted with an exogenous biomaterial.

In some embodiments, implanting an exogenous biomaterial stimulates immunogenicity by increasing human immune cell signaling relative to a control. Increased human immune may cell signaling may be increased cytokine production (e.g., IFNγ, TNFβ) and/or increased protein production (e.g., cell surface marker). Human immune cell signaling may be assessed by any method known in the art including, but not limited to: ELISAs to detect cytokines or other secreted proteins, radioimmunoassay to detect cytokines or other secreted proteins, and bioassays specific to a particular cytokine or protein In some embodiments, human immune cell signaling is increased by 5%-100%, 10%-95%, 15%-90%, 20%-85%, 25%-80%, 30%-75%, 35%-70%, 40%-65%, 45%-60%, or 50%-55% or more compared to a human immune cell not contacted with an exogenous biomaterial. In some embodiments, human immune cell signaling is increased by 5%, 10%, 15%, 20%, 25%, 30%, 35%, 40%, 45%, 50%, 55%, 60%, 65%, 70%, 75%, 80%, 85%, 90%, 95%, or 100% or more compared to a human immune cell not contacted with an exogenous biomaterial.

### ADDITIONAL EMBODIMENTS

1. A method, comprising:
(a) implanting an exogenous biomaterial into an immunodeficient mouse, wherein the mouse comprises human adaptive immune cells and human innate immune cells; and
(b) assessing fibrosis following implantation of the biomaterial.
   2. The method of paragraph 1, wherein the immunodeficient mouse lacks mature mouse T cells, B cells, and functional natural killer (NK cells) and is deficient in mouse cytokine signaling.
   3. The method of paragraph 1 or 2, wherein the immunodeficient mouse supports engraftment of CD33+ myeloid lineages.
   4. The method of any one of the preceding paragraphs, wherein the immunodeficient mouse is a non-obese diabetic (NOD) mouse.
   5. The method of any one of the preceding paragraphs, wherein the immunodeficient mouse comprises an inactivated Prkdc^{scid} allele and/or an inactivated mouse Il2rg allele.
   6. The method of any one of the preceding paragraphs, wherein the immunodeficient mouse comprises a transgene encoding a human hematopoietic cytokine.
   7. The method of paragraph 6, wherein the transgene encodes human colony stimulating factor 1 (CSF1).
   8. The method of paragraph 6, wherein the transgene encodes a cytokine selected from human stem cell factor (SCF), human granulocyte/macrophage-colony stimulating factor 2 (GM-CSF2), and human interleukin-3 (IL-3).
   9. The method of paragraph 8, wherein the immunodeficient mouse comprises a transgene encoding human SCF, a transgene encoding human GM-CSF2, and a transgene encoding IL-3.
   10. The method of any one of the preceding paragraphs, wherein the immunodeficient mouse is engrafted with human fetal tissue.
   11. The method of paragraph 10, wherein the human fetal tissue comprises tissue from human fetal bone marrow, liver, and/or thymus.
   12. The method of any one of the preceding paragraphs, wherein the immunodeficient mouse is engrafted with human hematopoietic stem cells.
   13. The method of paragraph 12, wherein the human fetal tissue is autologous with the human hematopoietic stem cells.
   14. The method of any one of the preceding paragraphs, wherein the immunodeficient mice comprise human myeloid cells.
   15. The method of any one of the preceding paragraphs, wherein the immunodeficient mice comprise human macrophages.
   16. The method of any one of the preceding paragraphs, wherein the biomaterial is a synthetic biomaterial.
   17. The method of any one of the preceding paragraphs, wherein the biomaterial comprises hydrogel alginate or polymer polydimethylsiloxane (PDMS).
   18. The method of any one of the preceding paragraphs, wherein the biomaterial is a biomedical device.
   19. The method of any one of the preceding paragraphs, wherein the biomaterial is implanted subcutaneously or intraperitoneally.
   20. The method of any one of the preceding paragraphs, wherein the assessing fibrosis occurs about 1 to 4 weeks following implantation of the biomaterial.

### EXAMPLES

### Example 1. Producing a Humanized Mouse Model of Fibrosis

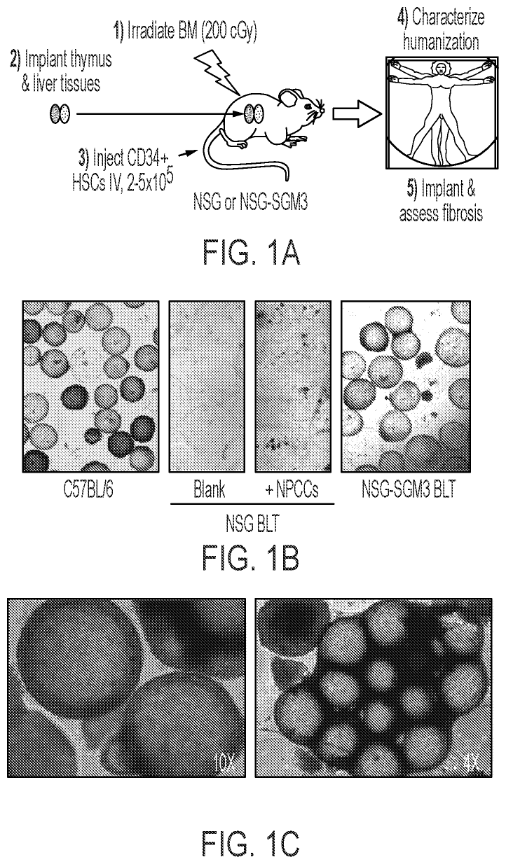

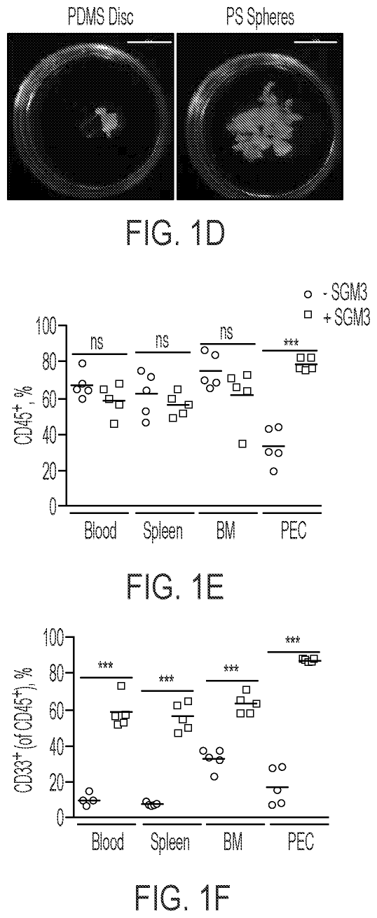

Humanized mice are severely immunodeficient mice that support engraftment with functional human immune systems following transplantation with human HSCs and/or tissues/organoids to assist human immune cell development (Shultz et al. (2012), Nature Reviews Immunology 12, 786-798). To develop a humanized model for the fibrotic response, NOD-scid mice also bearing a targeted mutation in the interleukin 2 (IL-2) receptor common gamma chain locus (IL2g^{null}), commonly abbreviated as NOD-scid-gamma (NSG)(Shultz et al., (2005), J Immunol 174, 6477-6489) as well as NSG mice that transgenically expressed human stem cell factor (SCF), GM-CSF, and IL3, abbreviated as NSG-SGM3 were studied. First, empty 500 μm diameter hydrogel spheres (0.5 ml/mouse) and similar spherical capsules containing a high load (10,000 clusters/mouse) of foreign xenogeneic neonatal porcine cell clusters (NPCCs) were transplanted into the IP cavity of both non-engrafted NSG mice and human fetal thymus and liver engrafted (BLT) mice that were also injected with autologous human CD34+ HSCs, termed “NSG-BLT” mice. (FIG. 1A).

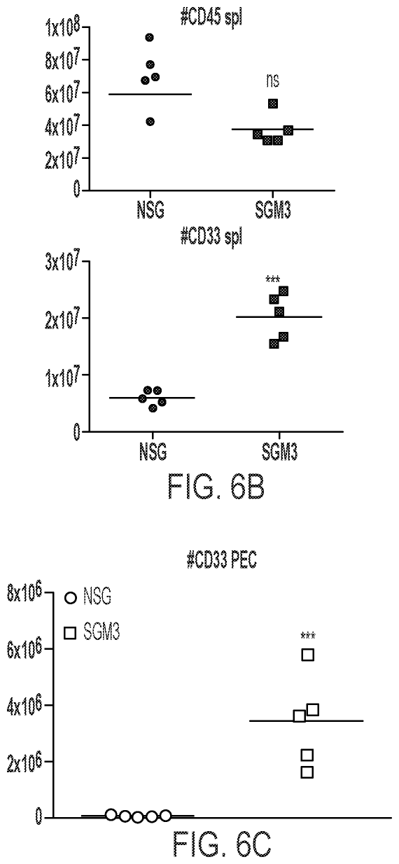

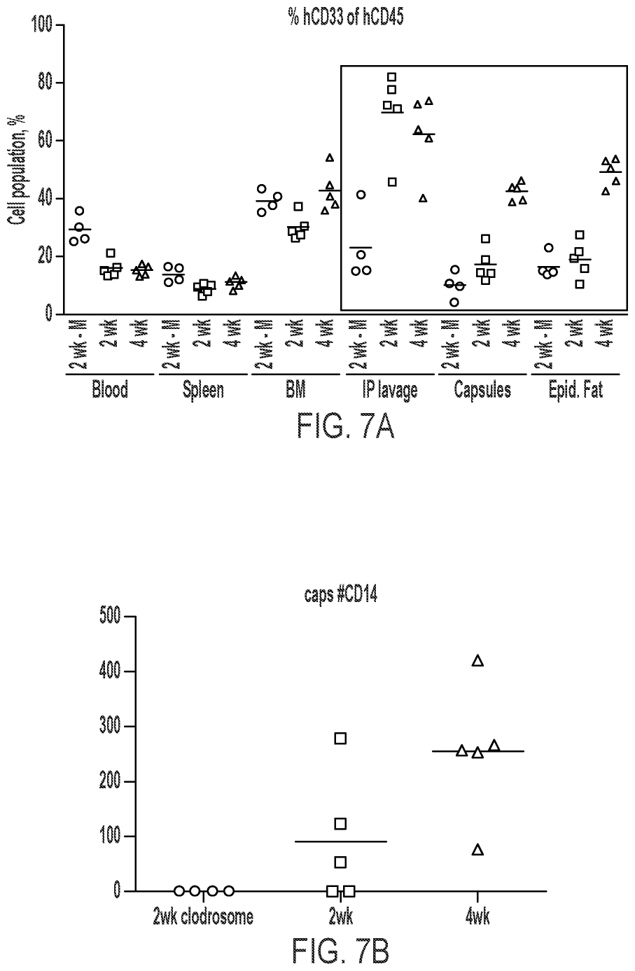

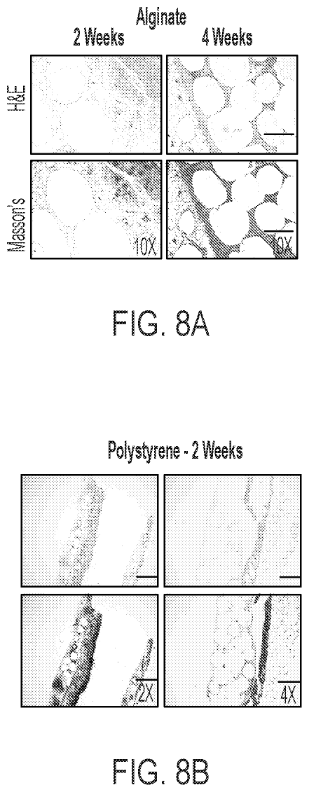

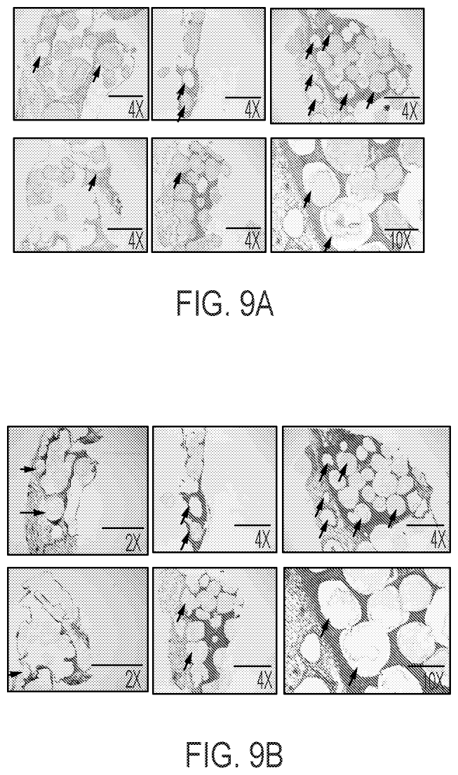

As expected, in non-engrafted NSG mice, no fibrotic response was observed after 4 weeks. However, as compared to fibrosed NPCC-containing 500 μm SLG20 alginate capsule controls retrieved from fibrosis-positive control C57BL/6 mice (FIG. 1B, left), even in NSG-BLT mice engrafted with a functional human immune system, no fibrotic response was observed after 4 weeks with either empty capsules or capsules containing NPCCs (FIG. 1B, middle). It was previously reported that a similar humanized NOD-scid model was used to test prevention of T cell-mediated rejection of exposed pig islet transplants, and to establish treatments to ensure long-term efficacy in the presence of a human immune system prior to transplantation into humans (Tonomura et al., (2008), Xenotransplantation 15, 129-135). However, this was not done in the presence of a biomaterial encapsulation system, suggesting that the fibrotic response may not be intact in that mouse model either. Lack of fibrosis was also observed for samples retrieved from another humanized variant, HSC-engrafted NRG-Akita mice (NOD.Cg-Rag1^{tm1Mom}Ins2^{Akita}Il2rg^{tm1Wjl}/SzJ) (Brehm et al., (2010), Diabetes 59, 2265-2270). However, while both models had appropriate levels of adaptive immune T and B cells, the innate immune arm of their immune systems were deficient. Therefore, to further enhance innate immune levels in the NSG-BLT model, NSG-SGM3 mice were used in order to stimulate development and maintenance of human immune myeloid cell populations. Only following adoption of the NSG-SGM3 BLT model (Billerbeck et al., (2011), Blood 117, 3076-3086) was an intact FBR against both NPCC-containing as well as blank (empty) alginate spheres observed (FIG. 1B; right, 1C; and 5A-5C). In addition, fibrosis of synthetic biomaterial polymer implants was observed in this model with both PDMS 5 mm molded discs and polystyrene 500 μm spheres after implantation for 4 weeks in the peritoneal cavity (FIG. 1D). When analyzing the human cell populations in the engrafted NSG-BLT mice, although human CD45⁺ immune cells were present throughout most of the mouse tissue compartments examined (blood, bone marrow, and spleen), few were present in the peritoneal cavity (˜5×10⁶ could be recovered from a peritoneal lavage) (FIGS. 1E and 6A-6B). Upon further inspection, NSG-SGM3 BLT mice exhibited human CD45⁺CD33⁺ innate myeloid cells in high numbers in circulating blood as well as spleen, bone marrow, and within the peritoneal exudate cell (PEC, ˜20×10⁶) population (FIGS. 1F and 6B-6C).

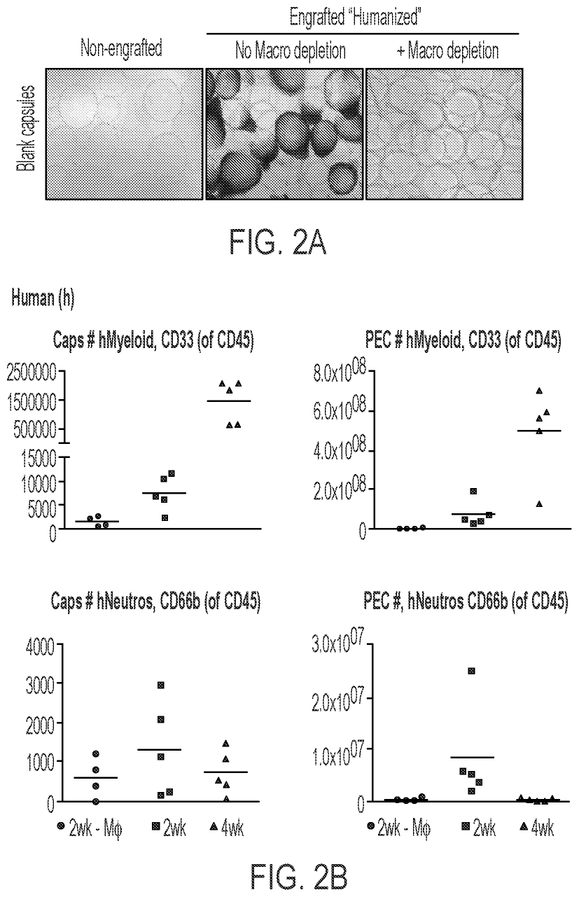

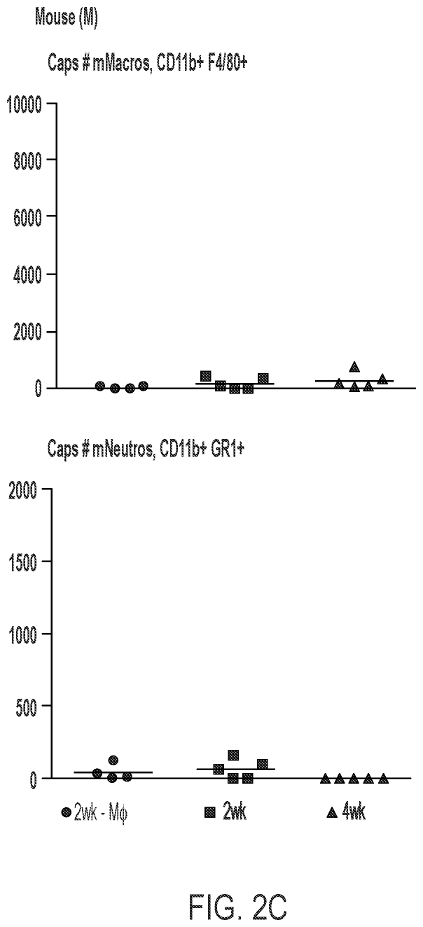

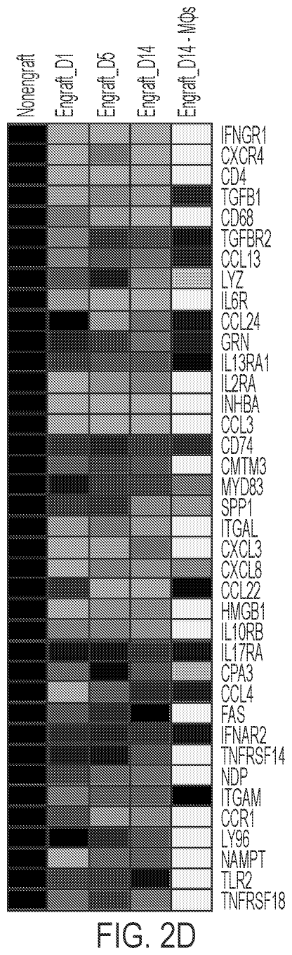

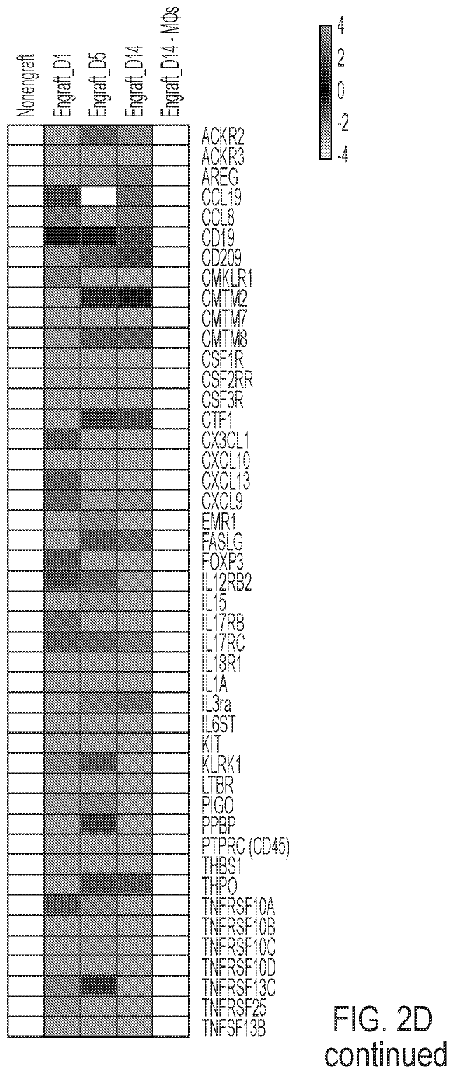

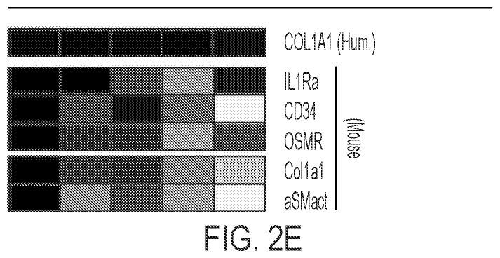

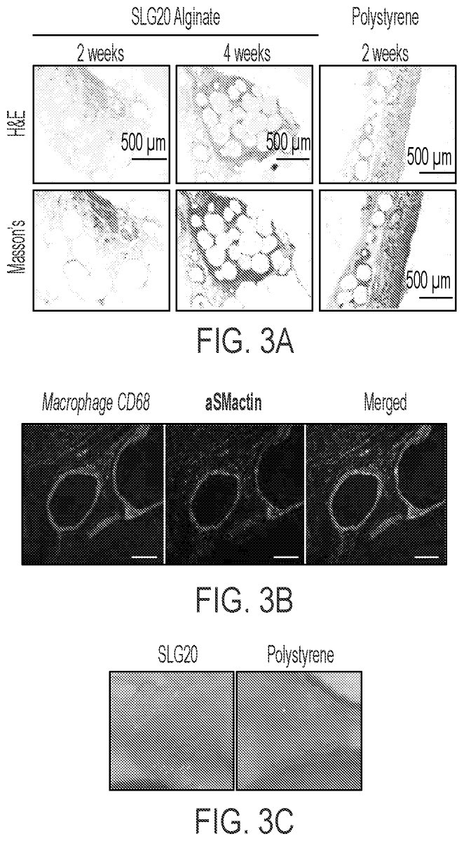

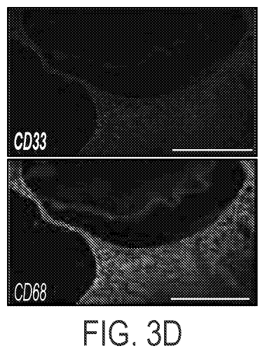

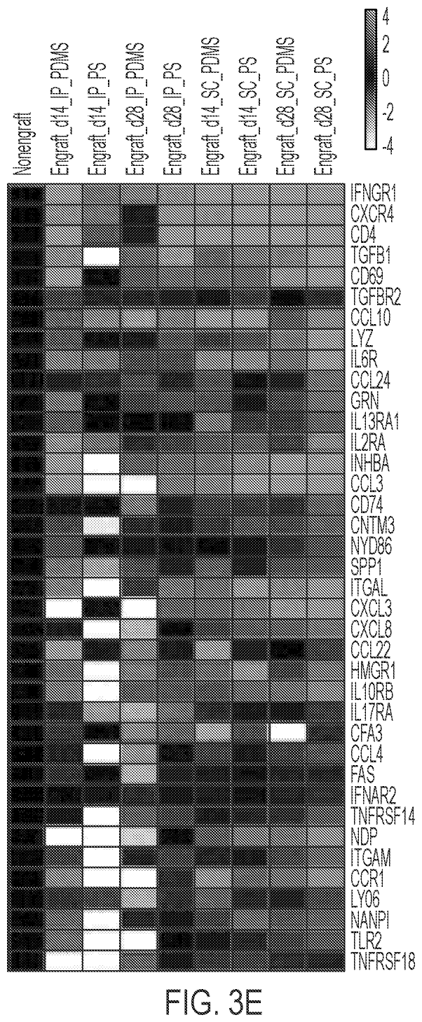

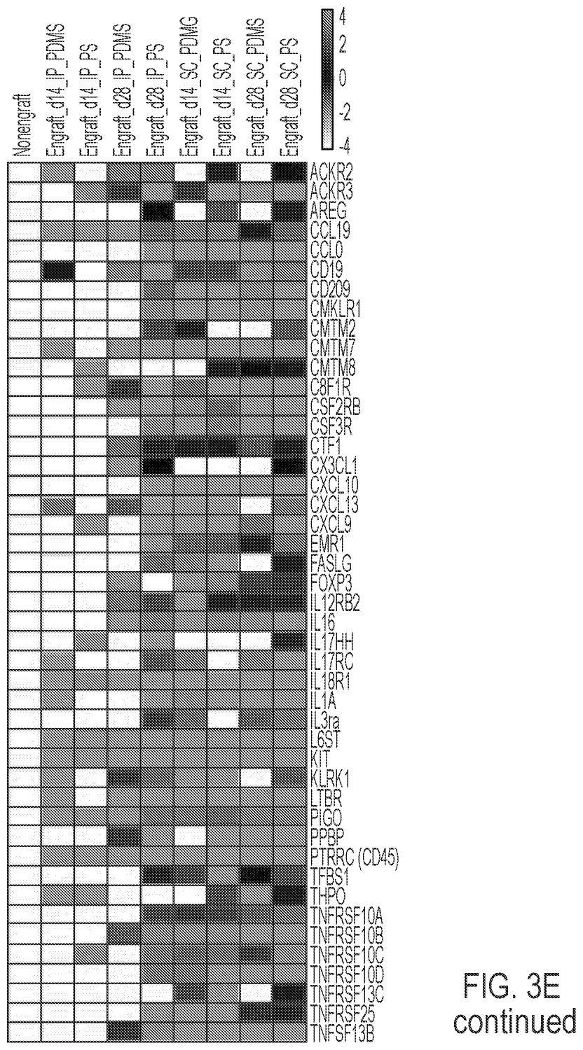

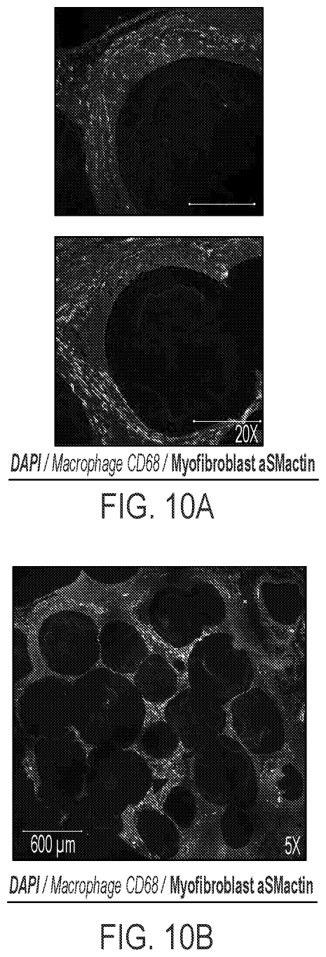

To confirm fibrosis induction was not occurring solely in the peritoneal space of engrafted NSG-SGM3 BLT mice, SLG20 alginate or polystyrene 500 μm spheres were implanted into the subcutaneous (SC) space. H&E and Masson's Trichrome staining of sections of excised SC tissue after 14 and 28 days confirmed that induction of FBR was not an IP space-specific phenomenon (FIG. 3A). Confocal microscopy also established that prominent foreign body giant cell (FBGC) formation (visualized by macrophage marker human CD68 staining, green) was intact in the humanized NSG-SGM3 BLT mouse model (FIGS. 3B, 10, and 11), despite historically being less apparent in mice, as opposed to NHPs and humans (Doloff et al.). In addition, FBGCs co-localized with an inner ring of myofibroblast marker αSMactin (FIG. 3B, red). Brightfield photos confirmed prominent fibrotic encapsulation of both alginate and polystyrene spheres upon retrieval following 4-week SC implantations (FIG. 3C). Immunofluorescent staining of immune cell infiltration zones immediately around SC implants was also used to confirm that the majority of earlier identified human CD33⁺ myeloid cells co-expressed human CD68, indicating that they are largely human innate immune macrophages (FIG. 3D). Additional analysis of RNA collected from dissociated tissues (IP & SC) taken at either 14 or 28 days post-implant confirmed that many of the same cell and cytokine markers identified from naturally derived biomaterial alginate hydrogel IP implants (as shown in FIG. 2) were also significantly increased for both synthetic PDMS disc and polystyrene (PS) sphere implants (FIG. 3E). These results collectively confirm that the human immune cell population(s) and signaling factors important in the fibrotic cascade in this humanized mouse model are quite similar to those observed in NHPs (Doloff et al.). Combined with the observation that foreign body giant cell (FBGC) formation, which is historically less apparent in mice but easily visible in both NHPs (Doloff et al.) and humans, is intact in the NSG-SGM3 BLT mice, these findings suggest that this new humanized model variant more closely mirrors observations seen in the clinic than do wildtype C57BL/6 mice.

### Example 2. Human Macrophages are Required to Produce Fibrosis in a Humanized Mouse Model

Previous work has examined fibrosis with wildtype C57BL/6 mouse and non-human primate models and noted the importance of macrophages in the fibrotic response in these animal systems (Veiseh et al.; Vegas et al.; Doloff et al.; Farah et al.; King et al., (2001) Journal of biomedical materials research 57, 374-383. To study the reliance on innate immune macrophages to drive the FBR in the NSG-SGM3 pro-fibrotic humanized mouse model, clodrosome-based depletion was performed (Doloff et al.). As expected, clodrosome depletion of human macrophages resulted in complete loss of FBR following SLG20 alginate 500 μm diameter sphere 2-week peritoneal implantations in engrafted NSG-SGM3 BLT mice (FIG. 2A). Flow cytometry analysis of cells dissociated from the surface of retrieved alginate spheres at 2 and 4 weeks post-peritoneal cavity implantation, and nearby epididymal fat pads, or taken by peritoneal lavage (FIGS. 2B, top, green and blue, respectively; and 7) showed significantly increased levels of human CD45⁺CD33⁺ myeloid cells, which were also eliminated by clodrosome-maintained macrophage depletion throughout the 2-week implantation period (FIG. 2B, top, red). By comparison, percentages of human neutrophils (CD45⁺CD66b⁺) did not significantly change in either case (FIG. 2B, bottom). The prior suppression of mouse immunity by irradiation was highly efficient, with absolute counts of residual mouse macrophages (F4/80⁺Cd11b⁺) and neutrophils (Ly6g⁺Cd11b⁺) being substantially reduced (FIG. 2C).

NanoString array-based analysis of human physical cell markers, cytokines, and cytokine receptors was performed on RNA extracts taken from cells dissociated directly off of the surface of retrieved alginate spheres collected at 1, 5, and 14 days post-implantation in engrafted NSG-SGM3 BLT mice, and compared to non-engrafted NSG-SGM3 controls and engrafted NSG-SGM3 BLT mice treated with macrophage-depleting clodrosome (FIG. 2D). Similar to wildtype FBR, not only macrophages (e.g., CD68, CSF1R, and EMR1-positive cells) but also B cells (e.g., CD19-positive cells) and macrophage-expressed B cell chemoattractant (Doloff et al.) CXCL13 were confirmed as some of the strongest dynamic cell and cytokine responders to biomaterial implantation. B cells have been previously implicated in potentiating fibrosis, perhaps due to their ability to regulate macrophage phenotype and activation (Affara et al., (2014), Cancer cell 25, 809-821). The chemokine CXCL13 has also been reported to be responsible for B cell recruitment in a model of lymphoid neogenesis (Carlsen et al., (2004) Blood 104, 3021-3027), and recently identified as a biomarker for idiopathic pulmonary fibrosis (Vuga et al., (2014), American journal of respiratory and critical care medicine 189, 966-974). Lastly, no human collagen (COL1A1) expression was observed; instead, a slightly delayed increase of both mouse (host) fibroblast marker uSMactin and fibrotic extracellular matrix (ECM) deposition marker collagen (Col1a1) was detected, indicating mouse myofibroblast crosstalk with the human immune system (FIG. 2E).

### Example 3. Mapping Human Immunologic Infiltration of Biomaterial Implants

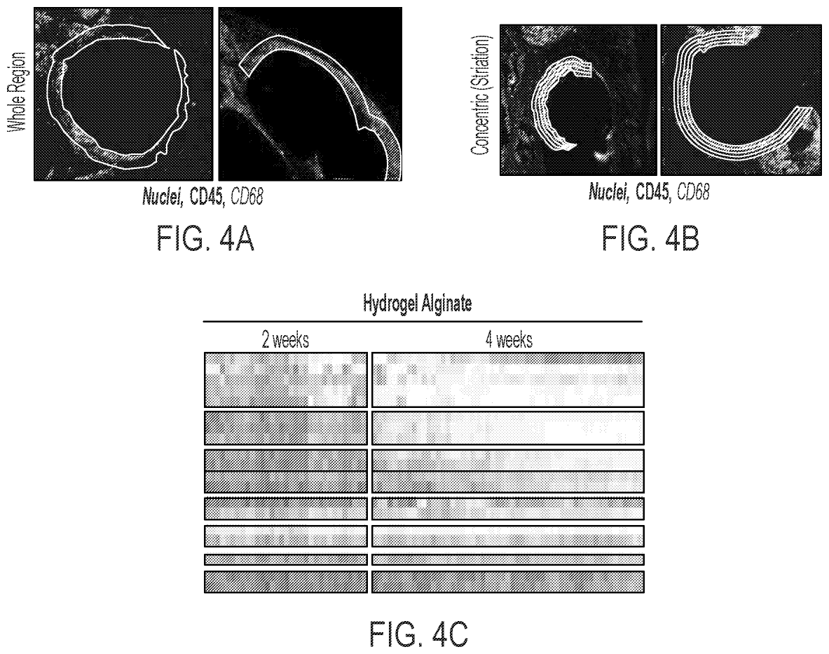

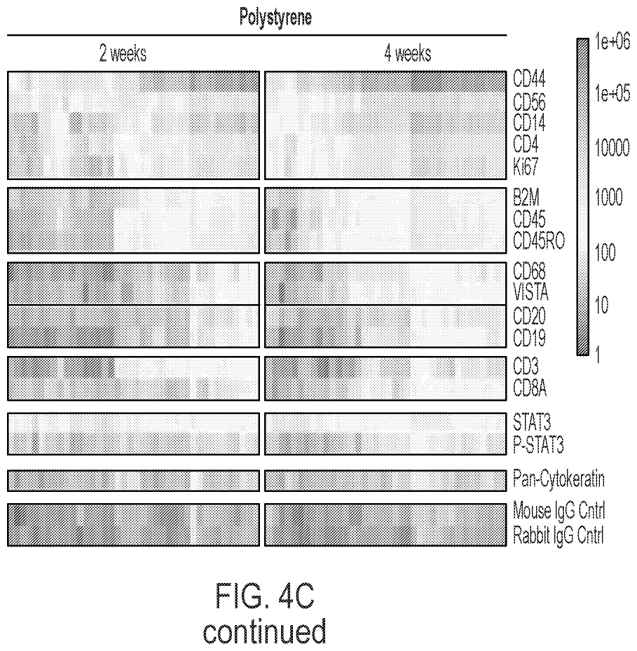

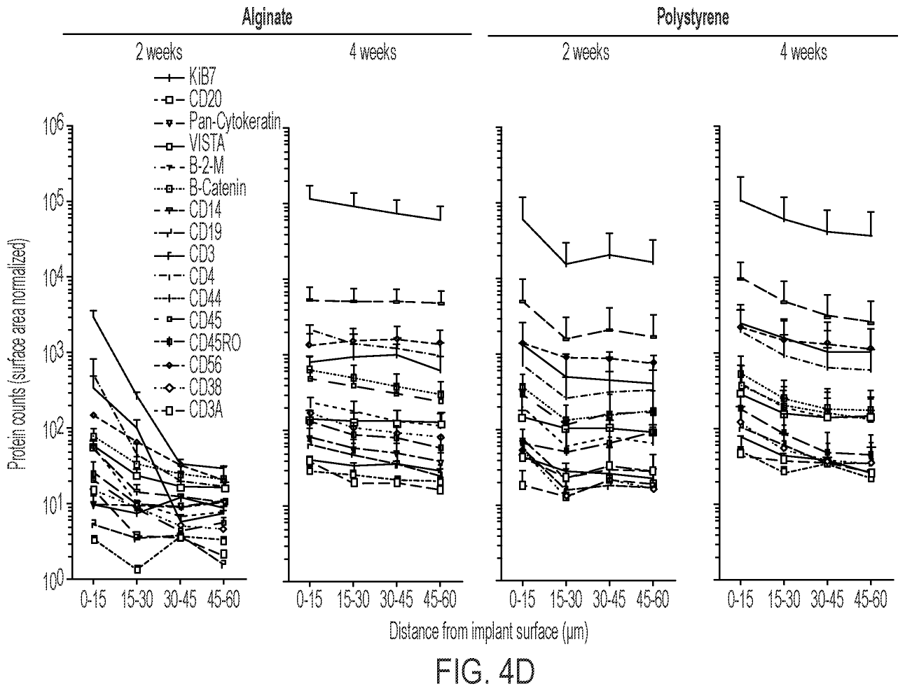

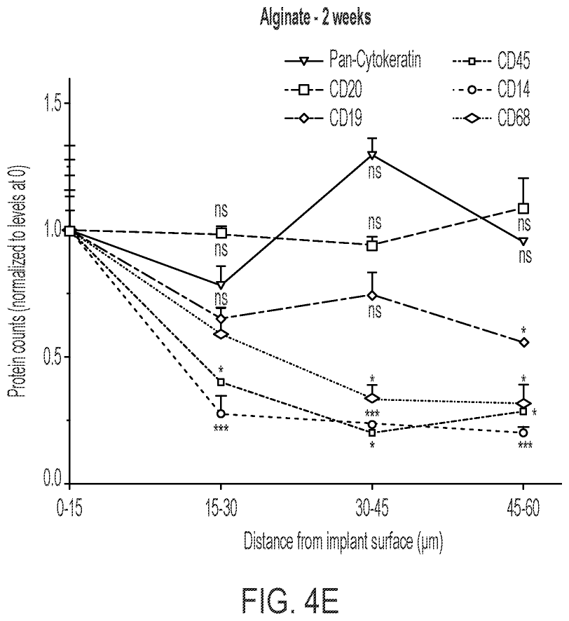

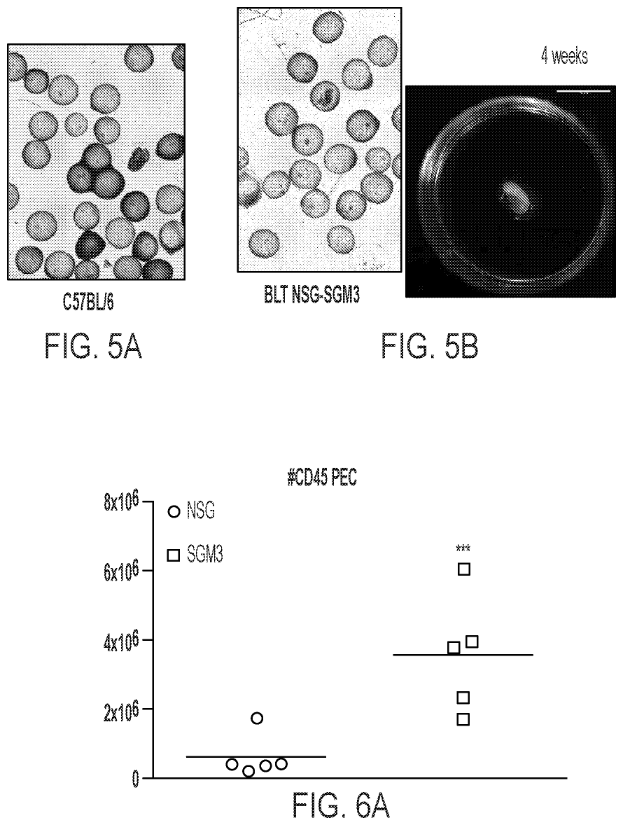

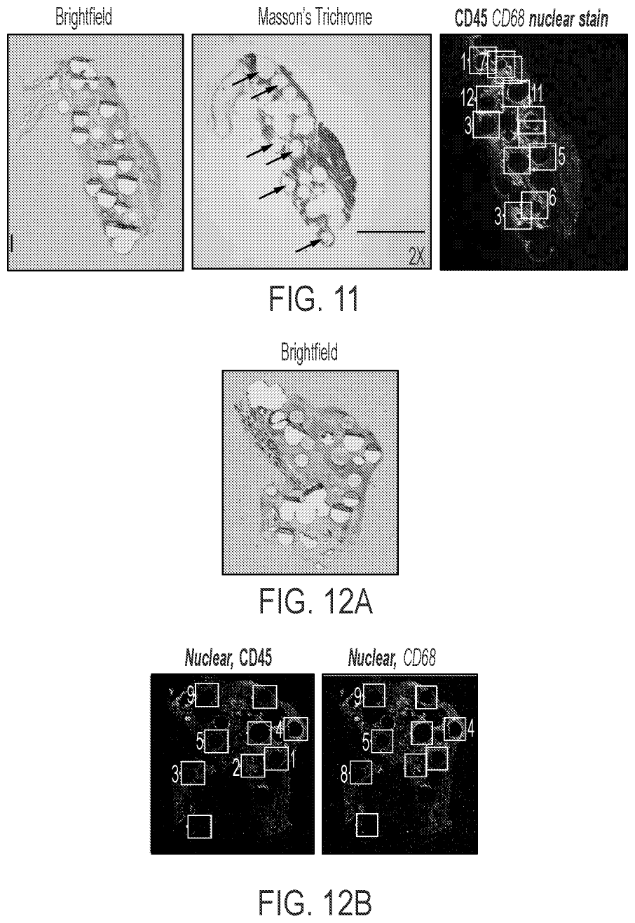

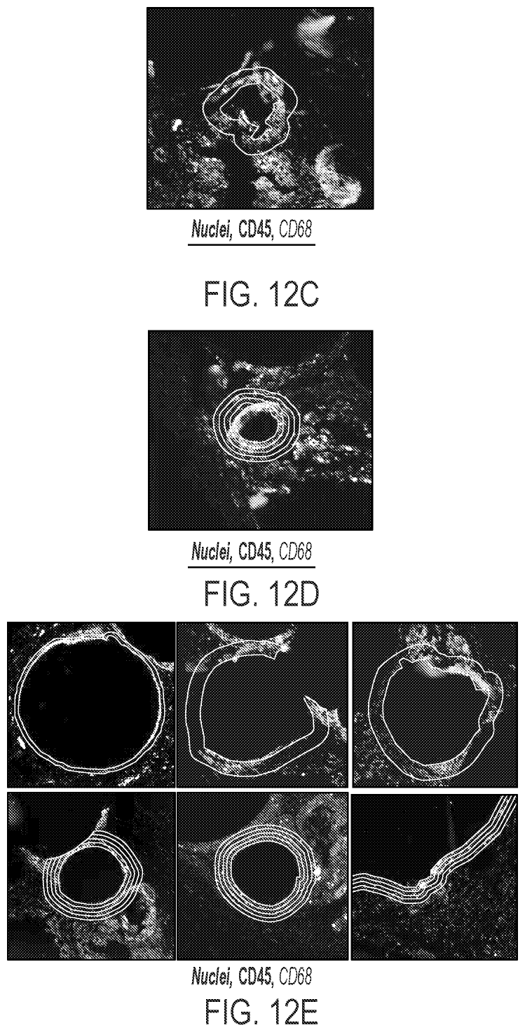

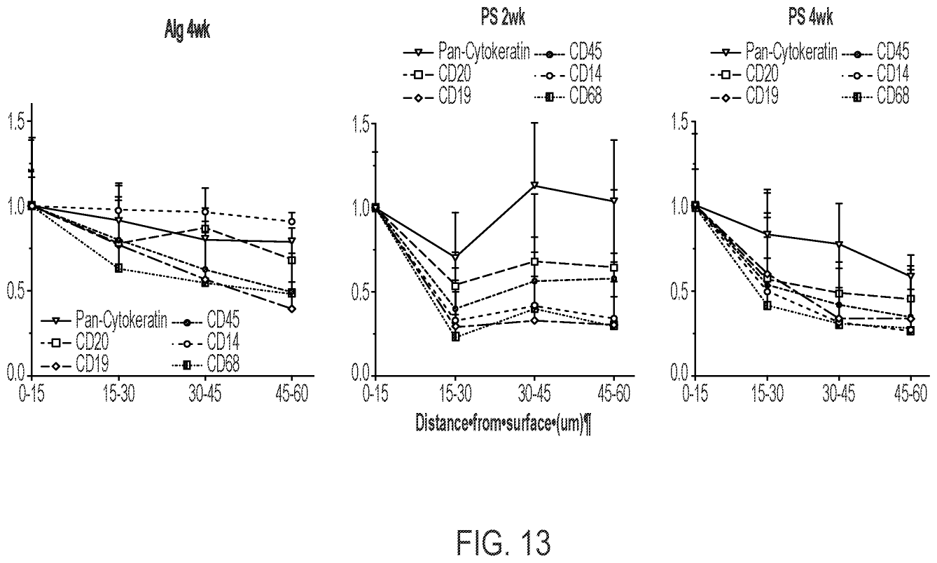

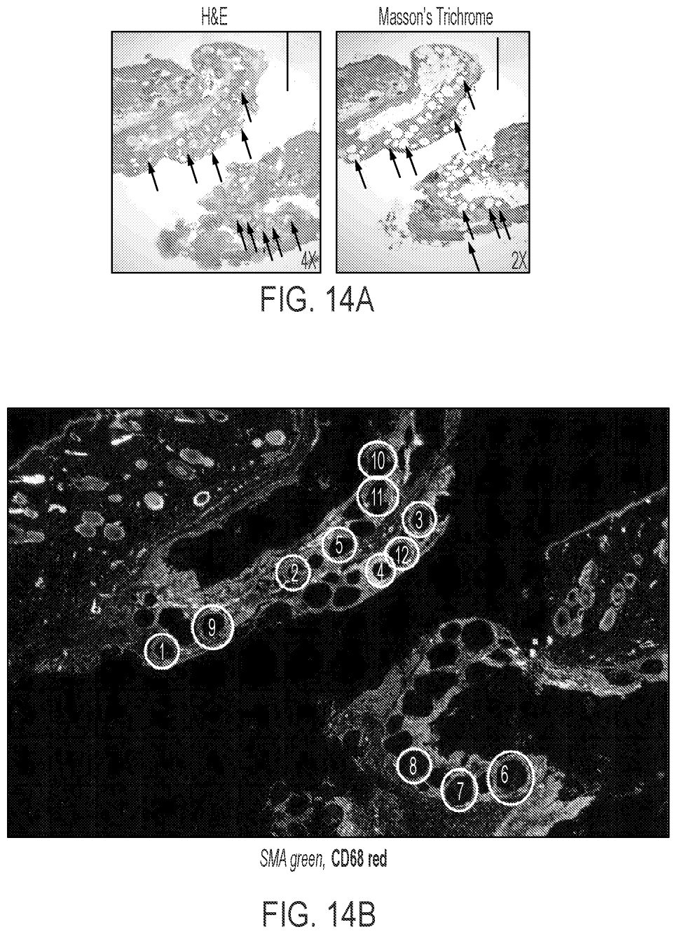

In order to further define the immunologic infiltration around biomaterial implants, a NanoString protein-level 3D Spatial Profiling (DSP) analysis platform was performed on human immune cell-mediated FBR around implanted biomaterials in engrafted NSG-SGM3 BLT mice. SLG20 alginate or polystyrene 500 μm diameter spheres were implanted into the subcutaneous (SC) space of NSG-SGM3 BLT mice for 14 and 28 days, and, per NanoString DSP protocols, immunofluorescence microscopy using DAPI nuclear (blue), human leukocyte marker CD45 (red), and human macrophage marker CD68 (green) staining was used to identify (whole) regions of interest (ROIs) (white lined areas) for subsequent UV laser ablation to decouple linked nucleotide probes to measure binding of a 30-plex human antibody panel (FIGS. 4A, 11, and 12A-12C). In discussion with NanoString, it was determined that ROIs could be performed around the same implant site but with successive rounds of ablation where the UV laser was incrementally increased 15 microns at a time, thereby capturing concentric rings of antibody binding and immune localization data as a function of distance moving away from the implant surface (FIGS. 4B and 12D-12E). Heat map plots as well as log-scale line graph depictions of changing antibody data as a function of distance were produced for the entire DSP NanoString human 30-plex antibody panel, with data for both earlier 2-week and later 4-week alginate and polystyrene implants (FIGS. 4C, 4D, and 13). While alginate had a more muted human immune response at 2 weeks (similar to reduced immunogenicity at early timepoints in wildtype C57BL/6 mice), the response was as robust as against polystyrene by 4 weeks (FIG. 4D, left vs. right). Furthermore, it was determined that macrophage marker for human (Hu) CD68 was largely localized directly at the implant surface and dropped off exponentially as distance from the implant increased, while B cell markers Hu CD19 and Hu CD20 decreased more slowly in a linear fashion moving away from the implant surface at early times for less immunogenic hydrogel alginate spheres (FIG. 4E), with CD19 localizing more towards the implant surface (FIG. 13). While the NanoString DSP platform was utilizable on human/humanized tissues, it was not the case with previously collected, in-house non-human primate samples (FIGS. 14A, 14B). Unfortunately, many of the antibodies in the 30-plex system showed poor signal detection in non-human primate model tissues, suggesting that use of the humanized mouse fibrosis model described herein will provide a better surrogate model over costly and animal facility prohibitive higher order NHP use, for which such spatial analysis methods are not yet optimal.

### Material and Methods

### A. Materials/Reagents

In vitro reagents were obtained from Life Technologies (Carlsbad, CA), unless otherwise noted. Antibodies: Alexa Fluor-conjugated anti-human CD45, CD14, CD33, and CD66b (described below) as well as anti-mouse F4/80 and Ly6g/Gr1 were purchased from BD Biosciences, Inc. (San Jose, CA, USA), eBiosciences (San Diego, CA, USA), BioLegend (San Diego, CA, USA), or Miltenyi Biotech (San Diego, CA, USA), as denoted below. For human/primate immunostaining, anti-human CD68 Alexa Fluor-conjugated (488 or 647) antibody was purchased from Santa Cruz (Dallas, TX). The same CD11b (anti-mouse/human) antibody (BioLegend) was used for both human and mouse staining. Cy3-conjugated anti-mouse alpha smooth muscle actin antibody was purchased from Sigma Aldrich (St. Louis, MO). Medium polystyrene spheres (mean 526.6 μm+/−53.3 μm) (Cat #:136, Lot #: 30055) were purchased from Phosphorex (Hopkinton, MA). Material aliquots used in this study were submitted for endotoxin testing by a commercial vendor (Charles River, Wilmington, MA) with results showing that spheres contained <0.05 EU/ml of endotoxin levels (below detectable limits) (Supplemental Table 1).

### B. Fabrication of Alginate Hydrogel Spheres

Alginate hydrogel spheres were made with an in-house customized electro-jetting system, as previously described (Doloff et al.). Spheres were made with a 1.4% solution of sterile alginate (PRONOVA SLG20, NovaMatrix, Sandvika, Norway) dissolved in 0.9% saline, and crosslinked with 250 mL of sterile 20 mM BaCl₂ solution. Alginate hydrogel 500 μm diameter spheres were generated with a 25G blunt needle, a voltage of 5 kV and a 200 μl/min flow rate. After, alginate spheres were washed with HEPES buffer 4 times and stored overnight at 4° C. Immediately prior to implantation, spheres were washed an additional 2 times with 0.9% saline.

### C. Implantation Surgeries

All protocols were approved by the UMass Medical School and MIT Committees on Animal Care, with all surgical procedures and post-operative care supervised by UMass and MIT veterinary staff. All wild type male immune-competent C57BL/6 mice or immunodeficient strains used for producing humanized models (described below) were ordered pathogen-free from The Jackson Laboratory (Bar Harbor, ME). Implanted mice were anesthetized with 3% isoflurane in oxygen and their abdomens were shaved and sterilized using betadine and isopropanol. Preoperatively, all mice received 0.05 mg/kg buprenorphine and 0.2 mL of 0.9% saline SC for pre-surgery analgesia and hydration. A midline incision (0.5 mm) was made and the peritoneal lining was exposed using blunt dissection. The peritoneal wall was then grasped with forceps and a 0.5-1 mm incision was made along the linea alba. 500 μL of spheres were then loaded into a sterile pipette and implanted into the intraperitoneal (IP) space. The incision was then closed using 5-0 PDS II absorbable sutures, and the skin was closed using wound clips and VetBond tissue glue. For subcutaneous implantation, ˜200-300 μL of 500 μm SLG20 or Polystyrene spheres were injected SC following anesthesia with isofluorane. For macrophage depletion, clodrosomes (200 μL/mouse) (Encapsula NanoSciences, Nashville, TN) were injected IP starting at −3 days (prior to implant day 0), and for every 7 days thereafter (on days −3, 4, and 11).

### D. Mice Used for Humanized Models

NOD.Cg-Prkdc^{scid}Il2rg^{tm1wji}/SzJ (NOD-scid IL2^{null}, NSG) mice, NOD.Cg-Prkdc^{scid} Il2rg^{tm1Wjl} Tg(CMV-IL3,CSF2,KITLG)1Eav/MloySzJ (NSG-SGM3) mice were obtained from The Jackson Laboratory (Bar Harbor, ME, USA). All mice were housed in a specific pathogen free facility in microisolator cages, given autoclaved food and maintained on sulphamethoxazole-trimethoprim medicated water (Goldline Laboratories, Ft Lauderdale, FL, USA) and acidified autoclaved water on alternate weeks. All experiments were performed in accordance with the guidelines of the Institutional Animal Care and Use Committee of the University of Massachusetts Medical School and the recommendations in the Guide for the Care and Use of Laboratory Animals (Institute of Laboratory Animal Resources, National Research Council, National Academy of Sciences, 1996).

### E. Engraftment of Mice with Human Hematopoietic Stem Cells and Generation of BLT Mice

Human fetal tissues (gestational age 16 to 20 weeks) were obtained from Advanced Bioscience Resources (Alameda, CA, USA). The tissues were washed with RPMI supplemented with penicillin G (100 U/ml), streptomycin (100 mg/ml), fungizone (0.25 ug/ml) and gentamycin (5 ug/ml), and 1 mm³ fragments of the fetal thymus and liver were implanted in the renal subcapsular space of 8 to 12 week old NSG and NSG-SGM3 mice that were previously irradiated with 200 and 100 cGy, respectively. Implanted mice were subsequently injected intravenously with 1×10⁵ CD34⁺ autologous fetal liver-derived HSCs (determined by flow cytometry with a CD34 specific antibody, clone 581, BD Biosciences) between 4 to 6 hours after irradiation, as described previously (Aryee et al., (2014), Methods Mol Biol. 1185, 267-278). Mice were injected subcutaneously with gentamycin (0.2 mg) and cefazolin (0.83 mg) post-surgery. After 12 weeks, flow cytometry analyses of the peripheral blood of HSC recipients determined the engraftment of the human immune system.

### F. Retrieval of Cells, Tissues, and Materials

At desired time points post-implantation, as specified in the figures, mice were euthanized by CO₂ administration, followed by cervical dislocation. In certain instances, 5 ml of ice cold PBS was first injected intraperitoneally to rinse out and collect free-floating IP immune cells. An incision was then made using forceps and scissors along the abdomen, and IP lavage volumes were pipetted out into fresh 15 ml falcon tubes (each prepared with 5 ml of RPMI cell culture media). Next, a wash bottle tip was inserted into the abdominal cavity. KREBS buffer was then used to wash out all material spheres into petri dishes for collection. After ensuring all the spheres were washed out or manually retrieved (if fibrosed directly to IP tissues, in particular epididymal and mesentery fat pads), they were transferred into 50 mL conical tubes for downstream processing and imaging. In certain instances, after IP lavage, portions of fibrosed IP tissues and material spheres were also excised for downstream flow cytometry and expression analyses.

### G. Imaging of the Retrieved Material Spheres

For phase contrast imaging retrieved materials were gently washed using Krebs buffer and transferred into 35 mm petri dishes for phase contrast microscopy using an EVOS XL microscope (Advanced Microscopy Group). For bright-field imaging of retrieved materials, samples were gently washed using Krebs buffer and transferred into 35 mm petri dishes for bright-field imaging using a Leica Stereoscopic microscope.

### H. Confocal Immunofluorescence

Immunofluorescent imaging was used to determine immune populations attached to spheres. Retrieved subcutaneous tissues were fixed using 4% paraformaldehyde at 4° C. Samples were then dehydrated and embedded in paraffin, sectioned and then permeabilized with 0.1% Triton×100, and blocked with 1% BSA for 1 hr. Next, spheres were incubated for 1 hr in an antibody cocktail solution consisting of DAPI (500 nM) and specific marker probes (1:200 dilution) in BSA. After staining, samples were washed and cover-slipped following addition of ProLong Gold Antifade Mountant (Thermo, Cat #P10144), and then imaged using a LSM 700 point scanning confocal microscope (Carl Zeiss Microscopy, Jena Germany) equipped with 5 and 10× objectives. For non-human primates (cynomolgus macaques), subcutaneous sphere-embedded samples were sectioned and stained according to traditional antigen retrieval and immunofluorescent methods, specifically looking at cellular nuclei (DAPI), macrophage marker CD68-AF488 (Santa Cruz, CA) and Cy3-conjugated anti-mouse alpha smooth muscle actin (fibrosis-associated myofibroblast marker) (Sigma Aldrich, St. Louis, MO).

### I. Histological Processing for H&E and Masson's Trichrome Staining

Retrieved material-containing tissue (intraperitoneal and/or subcutaneous) was fixed overnight using 4% paraformaldehyde at 4° C. After fixation, alginate sphere or retrieved tissue samples were washed using 70% alcohol. The materials were then paraffin embedded, sectioned at 5 μm thickness and stained according to standard histological (H&E or Masson's Trichrome) methods.

### J. Flow Cytometry Analysis

Tissue samples for flow cytometry were prepared from mice as described below. Single cell suspensions were prepared from bone marrow and spleen. Peritoneal exudate cells (PECs) were recovered from mice by flushing the peritoneal cavity using 5 ml of PBS. Whole blood was collected by sub-mandibular bleed or cardiac puncture into tubes containing 1,000 U of heparin sodium injection, USP (Pfizer Injectables, New York, NY). Single-cell suspensions of 1×10⁶ cells in 100 μl or 100 μl of whole blood were washed with fluorescence activated cell sorter (FACS) buffer (PBS supplemented with 2% FBS) and then incubated with rat anti-mouse CD16/CD32 (clone 2.4G2, BD Biosciences) to block Fc binding. Fluorescently conjugated antibodies were then added to the samples and incubated for 30 min at 4° C. in the dark. Stained cell suspensions were washed and fixed with 2% paraformaldehyde. Alternatively, blood or single-cell suspensions were treated with BD FACS Lysing Solution (BD Biosciences). Fixed samples were then suspended in FACS buffer and analyzed using a flow cytometry. At minimum of 50,000 events were acquired on LSRII or FACSCalibur instruments (BD Biosciences). Data analysis was performed with FlowJo (Tree Star, Inc., Ashland, OR, USA) software.

Single-cell suspensions of freshly excised tissues were prepared using a gentleMACS Dissociator (Miltenyi Biotec, Auburn, CA) according to the manufacturer's protocol and as previously described (Doloff et al.). Single-cell suspensions were prepared in a passive PEB dissociation buffer (1×PBS, pH 7.2, 0.5% BSA, and 2 mM EDTA) and suspensions were passed through 70 μm filters (Cat. #22363548, Fisher Scientific, Pittsburgh, PA). All tissue and material sample-derived, single-cell populations were then subjected to red blood cell lysis with 5 ml of 1×RBC lysis buffer (Cat. #00-4333, eBioscience, San Diego, CA, USA) for 5 min at 4° C. The reaction was terminated by the addition of 20 ml of sterile 1×PBS. The cells remaining were centrifuged at 300-400 g at 4° C. and resuspended in a minimal volume (˜50 μl) of eBioscience Staining Buffer (cat. #00-4222) for antibody incubation. All samples were then co-stained in the dark for 25 min at 4° C. with fluorescently tagged monoclonal antibodies. For analysis of human cell populations by flow cytometry, monoclonal antibodies (mAb) specific for mouse CD45 (clone 30-F11, BD Biosciences) and the following human antigens: CD14 (clone HCD14), CD33 (clone WM53), CD45 (clone 2D1), and CD66b (clone G10F5). For human/primate immunostaining, anti-human CD68 Alexa Fluor-conjugated (488 or 647) antibody was purchased from Santa Cruz (Dallas, TX). Antibodies for flow cytometry were purchased from BD Biosciences, Inc. (San Jose, CA, USA), eBiosciences (San Diego, CA, USA), BioLegend (San Diego, CA, USA), or Miltenyi Biotech (San Diego, CA, USA). For mouse, antibodies specific for the cell markers CD68 (1 μl (0.5 μg) per sample; CD68-Alexa647, Clone FA-11, Cat. #11-5931, BioLegend), F4/80 (1 μl (0.5 μg) per sample; F4/80-Alexa647, Clone BM8, Cat. #123122, BioLegend), Ly-6G (Gr-1) (1 μl (0.5 μg) per sample; Ly-6G-Alexa-647, Clone RB6-8C5, Cat. #108418, BioLegend), and/or CD11b (1 μl (0.2 μg) per sample; or CD11b-Alexa-488, Clone M1/70, Cat. #101217, BioLegend) were used. Two ml of eBioscience Flow Cytometry Staining Buffer (Cat. #00-4222, eBioscience) was then added, and the samples were centrifuged at 400-500 g for 5 min at 4° C. Supernatants were aspirated, and this wash step was repeated two more times with staining buffer. Following the third wash, each sample was resuspended in 300 μl of Flow Cytometry Staining Buffer and run through a 40 μm filter (Cat. #22363547, Fisher Scientific) for eventual FACS analysis using a LSRII (BD Biosciences, San Jose, CA, USA). For proper background and laser intensity settings, unstained, single antibody, and IgG (labeled with either Alexa-488 or Alexa-647, BioLegend) controls were also run.

### K. NanoString Gene Expression Analysis

RNAs for mock-implanted (saline) treated controls, or for 500 μm alginate or polystyrene sphere-bearing humanized mouse strains (n=5/group), were isolated from tissue samples taken at various time points after implantation, as described. In general, respective RNAs were quantified, normalized to the appropriate loading concentration (100 ng/μl), and then 500 ng of each sample was processed according to NanoString manufacturer protocols for expression analysis via our customized multiplexed human or mouse cytokine and cytokine receptor expression panels (also including major immune marker probes), used for both comparisons between implant sites (intraperitoneal and subcutaneous), early and late time points (2 and 4 weeks), and alginate vs. polystyrene sphere implantation. RNA levels (absolute copy numbers) were obtained following nCounter (NanoString Technologies Inc., Seattle, WA) quantification, and group samples were analyzed using nSolver analysis software (NanoString Technologies Inc., Seattle, WA).

### L. Nanostring Digital Spatial Profiling (DSP) Analysis

An automated setup capable of imaging and sample collection was developed by modifying a standard microscope. For protein detection, a multiplexed cocktail of primary antibodies, each with a unique, UV photocleavable indexing oligo, and/or 1-3 fluorescent markers (antibodies and/or DNA dyes) was applied to a slide-mounted FFPE tissue section. The tissue slide was placed on the stage of an inverted microscope. A custom gasket was then clamped onto the slide, allowing the tissue to be submerged in 1.5 mL of buffer solution. The microcapillary tip is connected to a syringe pump primed with buffer solution, allowing for accurate aspiration of small volumes (<2.5 μL). Under the microscope, wide field fluorescence imaging was performed with epi-illumination from visible LED light engine. The tissue area of interest was then located using fluorescence imaging. 20× image corresponds to 650 μm×650 μm of tissue area with a CMOS camera. The 20× images were stitched together to yield a high-resolution image of the tissue area of interest. The regions of interest (ROIs) were then selected based on the fluorescence information and sequentially processed by the microscope automation. The steps performed for each ROI by the microscope automation were as follows: First, the microcapillary tip was washed by dispensing clean buffer out the capillary and into a wash station. Next, the tissue slide was bulk washed by exchanging the buffer solution on the slide via the inlet and outlet wash ports on the gasket clamp. The microcapillary tip was then moved into position directly over the ROI with a distance of 50 μm from the tissue. The local region of tissue around the ROI was washed by dispensing 100 μL of buffer solution from the microcapillary. Then, the area of tissue within the ROI was selectively illuminated with UV light to release the indexing oligos. UV LED light was collimated to be reflected from the DMD surface into the microscope objective, and focused at the sample tissue. Each micro mirror unit in the DMD corresponds to ˜1 μm² area of sample and reflects the UV light in controlled pattern based on the ROI selection in the image. Following each UV illumination cycle, the eluent was collected from the local region via microcapillary aspiration and transferred to an individual well of a microtiter plate or strip tubes. Once all ROIs were processed, indexing oligos were hybridized to NanoString optical barcodes for ex situ digital counting and subsequently analyzed with an nCounter® Analysis System.

Overview of Digital Spatial Profiling workflow:

(a) Molecular profiling of antibodies with conjugated photocleavable oligos and the nCounter system. (b) Overview of in situ protein profiling workflow. (1) Process: FFPE slide-mounted tissue is incubated with a cocktail of primary antibodies conjugated with DNA oligos via a photocleavable linker together with visible wavelength-imaging reagents. (2) View: Regions of interest (ROIs) are identified with visible light-based imaging reagents at low-plex to establish overall “architecture” of sphere slice (e.g. image nuclei and perhaps one or two key immune biomarkers). (3) Profile: Select ROIs are chosen for high-resolution multiplex profiling, and oligos from the selected region are released upon exposure to UV light. (4) Plate: Free photocleaved oligos are then collected via a microcapillary tube and stored in a microplate well for subsequent quantitation. (5) Digitally count: During the digital counting step, photocleaved oligos from the spatially-resolved ROIs in the microplate are hybridized to 4-color, 6-spot optical barcodes, enabling up to ˜1 million digital counts of the protein targets (distributed across all targets) in a single ROI using standard NanoString nCounter instruments.

### M. Statistical Analysis

Data are expressed as mean±SEM, and n=5 mice per time point and per treatment group. These sample sizes were chosen based on previous literature and for required power analysis to ascertain statistical significance while reducing mouse numbers as much as possible. All animals were included in analyses except in instances of unforeseen sickness or morbidity. Animal cohorts were randomly selected. Investigators were not blind to performed experiments. For FACS, data were analyzed for statistical significance either by unpaired, two-tailed t-test, or one-way ANOVA with Bonferroni multiple comparison correction, unless indicated otherwise, as implemented in GraphPad Prism 8; *: p<0.05 and ***: p<0.0001. For Nanostring, data was normalized using the geometric means of the NanoString positive controls and background levels were established using the means of the negative controls. Variance was similar between all compared groups. Housekeeping genes Tubb5, Hprt1, Bact, and Cltc were used to normalize between samples. Data was then log-transformed.

### N. Endotoxin and Glucan Testing Results

Endotoxin and glucan results for materials utilized in this study are provided in Table 1 below. Determined by Charles River Laboratory submission, and in-house testing. E. coli and Limulus Amebocyte Lysates were used as positive controls for general endotoxin. Such negative results have been corroborated previously by our group, and previously published in multiple studies (Kurtz et al., Med et al., Anderson et al., Wynn et al., Veiseh et al.). BDL=below detectable limits.

TABLE 1

| Sample | Endotoxin Test | Glucan Test |
|---|---|---|
| Saline control | <0.05 EU/mL (BDL) | <10 ρg/ml (BDL) |
| SLG20 alginate 500 | <0.05 EU/mL (BDL) | <10 ρg/ml (BDL) |
| μm spheres | | |
| Polystyrene spheres | <0.05 EU/mL (BDL) | <10 ρg/ml (BDL) |

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A method, comprising:
(a) implanting an exogenous biomaterial into an immunodeficient mouse, wherein the mouse comprises human adaptive immune cells and human innate immune cells; and
(b) assessing fibrosis following implantation of the biomaterial.

2. The method of claim 1, wherein the immunodeficient mouse lacks mature mouse T cells, B cells, and functional natural killer (NK cells) and is deficient in mouse cytokine signaling.

3. The method of claim 1 or 2, wherein the immunodeficient mouse supports engraftment of CD33+ myeloid lineages.

4. The method of any one of the preceding claims, wherein the immunodeficient mouse is a non-obese diabetic (NOD) mouse.

5. The method of any one of the preceding claims, wherein the immunodeficient mouse comprises an inactivated Prkdc^{scid} allele and/or an inactivated mouse Il2rg allele.

6. The method of any one of the preceding claims, wherein the immunodeficient mouse comprises a transgene encoding a human hematopoietic cytokine.

7. The method of claim 6, wherein the transgene encodes human colony stimulating factor 1 (CSF1).

8. The method of claim 6, wherein the transgene encodes a cytokine selected from human stem cell factor (SCF), human granulocyte/macrophage-colony stimulating factor 2 (GM-CSF2), and human interleukin-3 (IL-3).

9. The method of claim 8, wherein the immunodeficient mouse comprises a transgene encoding human SCF, a transgene encoding human GM-CSF2, and a transgene encoding IL-3.

10. The method of any one of the preceding claims, wherein the immunodeficient mouse is engrafted with human fetal tissue.

11. The method of claim 10, wherein the human fetal tissue comprises tissue from human fetal bone marrow, liver, and/or thymus.

12. The method of any one of the preceding claims, wherein the immunodeficient mouse is engrafted with human hematopoietic stem cells.

13. The method of claim 12, wherein the human fetal tissue is autologous with the human hematopoietic stem cells.

14. The method of any one of the preceding claims, wherein the immunodeficient mice comprise human myeloid cells.

15. The method of any one of the preceding claims, wherein the immunodeficient mice comprise human macrophages.

16. The method of any one of the preceding claims, wherein the biomaterial is a synthetic biomaterial.

17. The method of any one of the preceding claims, wherein the biomaterial comprises hydrogel alginate or polymer polydimethylsiloxane (PDMS).

18. The method of any one of the preceding claims, wherein the biomaterial is a biomedical device.

19. The method of any one of the preceding claims, wherein the biomaterial is implanted subcutaneously or intraperitoneally.

20. The method of any one of the preceding claims, wherein the assessing fibrosis occurs about 1 to 4 weeks following implantation of the biomaterial.
